(19) United States
(12) Patent Application Publication
(10) Pub. No.: US 2010/0113889 A1
(43) Pub. Date: May 6, 2010

(54) CARDIAC RISK STRATIFICATION UTILIZING BAROREFLEX SENSITIVITY MEASUREMENT

(75) Inventor: Raja N. Ghanem, Edina, MN (US)

Correspondence Address: Medtronic, Inc., 710 Medtronic Parkway, Minneapolis, MN, 55432, US

(73) Assignee: Medtronic, Inc.

(21) Appl. No.: 12/261,746

(22) Filed: Oct. 30, 2008

(51) Int. Cl. A61B 5/0456; A61B 5/02; A61B 5/0215

(52) U.S. Cl. 600/301; 600/508; 600/485

(57) ABSTRACT

A monitoring or therapy system may obtain a baroreflex sensitivity (BRS) measurement via an implantable medical device (IMD). The monitoring or therapy system then may generate a risk stratification indicator based on the BRS measurement. In some examples, the IMD generates the risk stratification indicator, while in other examples, an external computing device, such as a programmer, generates the risk stratification indicator. The monitoring or therapy system also may obtain at least one of a heart rate variability (HRV) measurement and a non-sustained ventricular tachycardia (NSVT) indicator via the IMD, and may generate the risk stratification indicator based on the BRS measurement and one or both of the HRV measurement and the NSVT indicator. In some examples, the monitoring or therapy system may generate an instruction, indicator, or alert based on the risk stratification indicator. The indicator may indicate that the patient is a candidate for an implantable therapy device.

### TECHNICAL FIELD

The present disclosure relates to implantable medical devices and, more particularly implantable medical devices for analysis of cardiac function in a patient.

### BACKGROUND

Congestive heart failure is a serious condition affecting a large number of patients. Patients diagnosed with congestive heart failure have a poor long-term prognosis in the absence of cardiac rhythm disease management therapy. The average life expectancy of a person suffering from congestive heart failure is approximately five years. Autonomic markers such as heart rate variability (HRV) and baroreflex sensitivity (BRS) can be useful in analyzing cardiac function.

HRV refers to changes in the variability of RR intervals, i.e., the interval between successive R waves indicating ventricular depolarization. BRS provides a measure of the ability of a patient's heart to react to changes in blood pressure by changing heart rate. Typically, BRS characterizes the relationship between systolic blood pressure and RR interval.

Several methods may be used to measure BRS, such as bolus injection of vasoactive drugs (e.g., phenylephrine), the Valsalva maneuver, and mechanical alteration of transmural carotid sinus pressure by means of the neck chamber. Such techniques generally provide BRS results that represent a snapshot in time and are compared against test results of other patients in order to determine whether the BRS indicates a heart failure condition.

### SUMMARY

In general, the disclosure is directed to techniques for generating a risk stratification indicator based on a BRS measurement that is computed using physiological parameters sensed by an implantable medical device (IMD). In some examples, the BRS measurement may be computed by the IMD based on the physiological parameters. In other examples, the IMD may sense the physiological parameters, and transmit data representative of the parameters to an external computing device, such as an IMD programmer, which then computes the BRS measurement. Examples of physiological parameters include parameters associated with blood pressure signals and cardiac signals.

The IMD or external computing device may generate the risk stratification indicator based on the BRS measurement. The risk stratification indicator may indicate the risk of cardiac arrhythmia or cardiac mortality to the patient. In this manner, the patient can be classified into one of several cardiac arrhythmia or cardiac mortality risk strata. In some examples, the risk stratification indicator may prompt a clinician to prescribe new or additional cardiac therapy, such as implantation of an IMD or delivery of a drug, or to adjust existing cardiac therapy, such as one or more parameters associated with cardiac electrical stimulation therapy or dosages associated with a drug.

In other examples, the IMD or external computing device may automatically generate an indicator based on the risk stratification indicator. The indicator may include, for example, an implantation indicator, which indicates that the patient is a candidate for implantation of an implantable therapy device, such as an implantable cardioverter/defibrillator (ICD), or an implantable drug delivery device. A patient may be considered a candidate for implantation of the device if the risk stratification indicator indicates, for example, that the patient is critically in need of the device or would generally benefit from the device in order to reduce cardiac arrhythmia or cardiac mortality risk. The IMD or external computing device may also initiate, cease, or adjust an existing cardiac therapy based on the risk stratification indicator. In some examples, risk stratification may be based not only on the BRS measurement, but also other information such as an HRV measurement, a non-sustained ventricular tachycardia (NSVT) indicator, an ejection fraction measurement, age, gender, history of heart failure or cardiac disease.

In one aspect, the disclosure is directed to a method comprising obtaining a baroreflex sensitivity (BRS) measurement for a patient via an implantable medical device (IMD), and generating a risk stratification indicator based on the BRS measurement, wherein the risk stratification indicator classifies the patient into one of a plurality of cardiac arrhythmia or cardiac mortality risk categories.

In another aspect, the disclosure is directed to an implantable medical device (IMD) comprising a measurement unit configured to obtain a baroreflex sensitivity (BRS) measurement for a patient, and a processor that generates a risk stratification indicator based on the BRS measurement, wherein the risk stratification indicator classifies the patient into one of a plurality of cardiac arrhythmia or cardiac mortality risk categories.

In another aspect, the disclosure is directed to a system comprising an implantable medical device (IMD) configured to obtain a baroreflex sensitivity (BRS) measurement for a patient, and an external computing device that receives the BRS measurement from the IMD, generates a risk stratification indicator based on the BRS measurement, wherein the risk stratification indicator classifies the patient into one of a plurality of cardiac arrhythmia or cardiac mortality risk categories.

In another aspect, the disclosure is directed to a computer readable medium comprising instructions that cause a programmable processor to receive a cardiac signal of a patient and a blood pressure signal of the patient via a measurement unit of an implantable medical device (IMD), determine a baroreflex sensitivity (BRS) measurement based on the cardiac signal and the blood pressure signal, and generate a risk stratification indicator based on the BRS measurement, wherein the risk stratification indicator classifies the patient into one of a plurality of cardiac arrhythmia or cardiac mortality risk categories.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is a conceptual diagram illustrating an example cardiac monitoring system.

FIG. 2 is a conceptual diagram illustrating an example cardiac therapy system.

FIG. 3 is a functional block diagram of an example implantable medical device that monitors a cardiac signal and a blood pressure signal.

FIG. 4 is a functional block diagram of an example implantable medical device that monitors a cardiac signal and blood pressure signal and provides stimulation therapy to a heart.

FIG. 5 is a functional block diagram of an example medical device programmer.

FIG. 6 is a flow diagram illustrating an example technique for generating a risk stratification indicator.

FIG. 7 is a flow diagram illustrating further detail of an example technique for obtaining a BRS measurement.

FIG. 8 is a flow diagram illustrating further detail of an example technique for generating a risk stratification indicator.

FIG. 9 is a flow diagram illustrating another example technique for generating a risk stratification indicator.

FIG. 10 is a flow diagram illustrating another example technique for generating a risk stratification indicator.

FIG. 11 is a block diagram illustrating an example system that includes an external device, such as a server, and one or more computing devices that are coupled to the IMD and programmer shown in FIG. 1 via a network.

### DETAILED DESCRIPTION

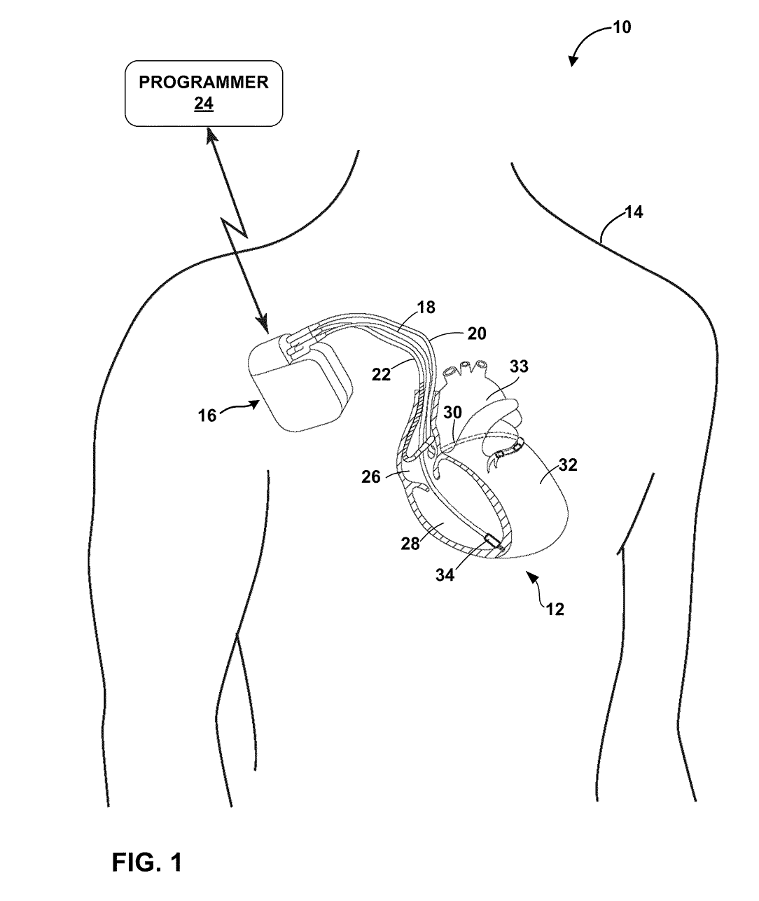

FIG. 1 is a conceptual diagram illustrating an example monitoring system 10 that may be used to obtain a baroreflex sensitivity (BRS) measurement for a patient 14 and generate a risk stratification indicator based on the BRS measurement. The risk stratification indicator classifies a patient into one of a plurality of cardiac arrhythmia or cardiac mortality risk categories. Monitoring system 10 may obtain the BRS measurement based on one or more detected physiological parameters of patient 14, such as blood pressure of patient 14 or cardiac signals of a heart 12 of patient 14. Patient 14 ordinarily, but not necessarily, will be a human. Monitoring system 10 includes an implantable medical device (IMD) 16, which is coupled to leads 18, 20, and 22, and a programmer 24.

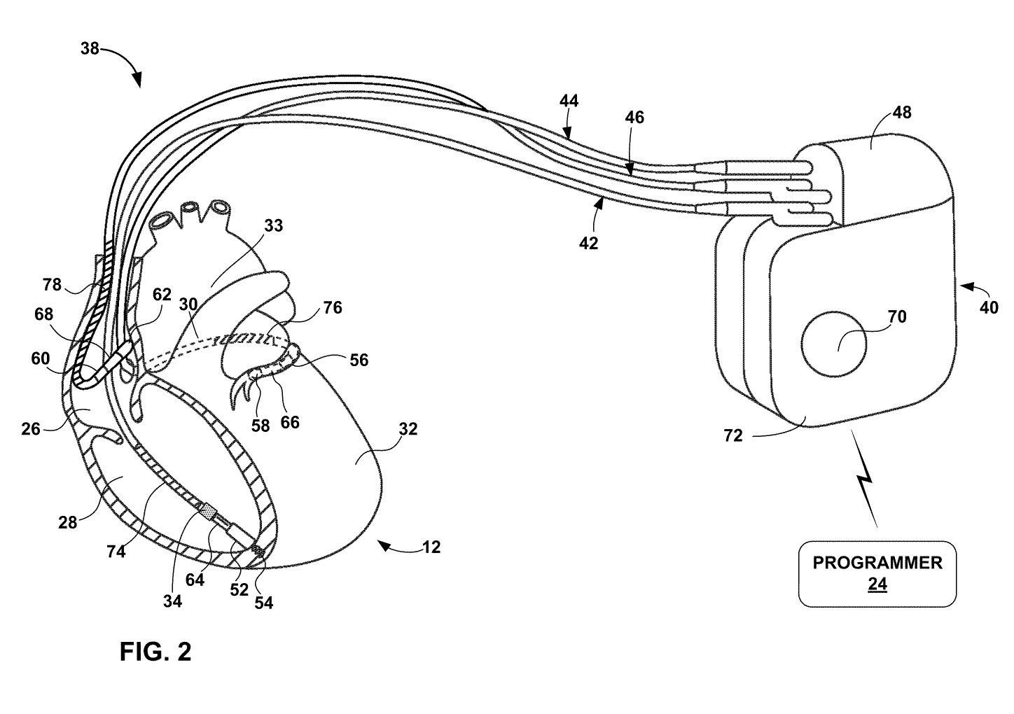

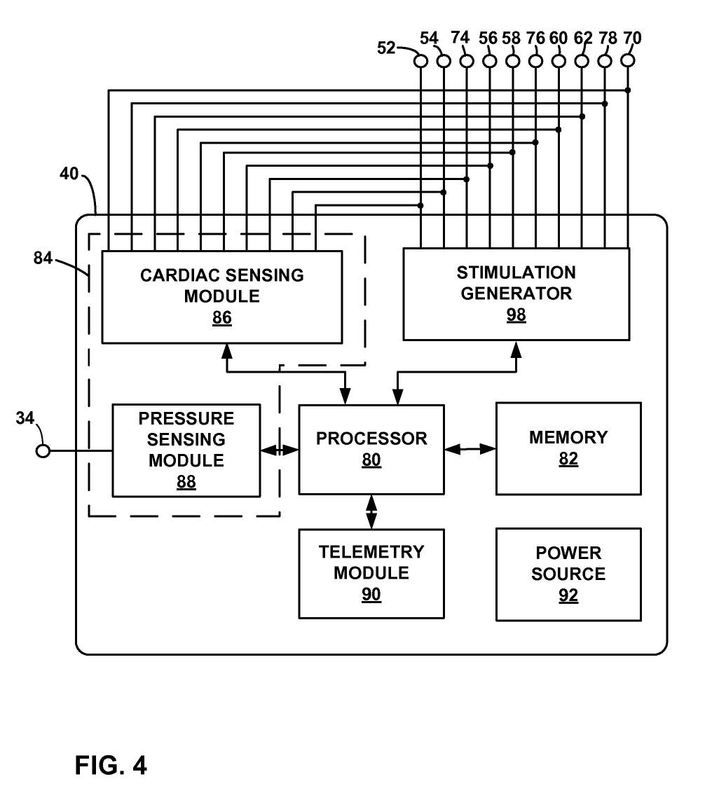

IMD 16 may be referred to as an implantable monitor or an implantable hemodynamic monitor (IHM). IMD 16 may be, for example, an implantable cardiac monitor that does not provide therapy (e.g., stimulation therapy) to patient 14. In this case, the IHM may be used to generate a risk stratification indicator to determine whether the patient is a candidate for implantation of an implantable therapy device, such as a cardiac pacemaker, an implantable cardioverter-defibrillator (ICD), or a cardiac resynchronization therapy (CRT) pacing device. Hence, in some examples, an IHM may be used in patient 14 in advance of implantation of a stimulation therapy device to determine whether implantation of a stimulation therapy device would be advisable for the patient. In other examples, an IHM may be used in conjunction with an implantable cardiac pacemaker to determine whether patient 14 may benefit from implantation of an ICD. An IHD may sense and process signals such as left ventricular and right ventricular pressure. In still other examples, e.g., as described with respect to FIGS. 2 and 4, IMD 16 may be incorporated in an implantable medical device that delivers electrical stimulation to heart 12 of patient 14. Examples of IMDs for delivery of electrical stimulation include a cardiac pacemaker, an ICD, or a CRT device, each of which provides electrical stimulation pulses and/or shocks to heart 12 via electrodes coupled to one or more leads.

Leads 18, 20, 22 extend into the heart 12 of patient 16 to sense electrical activity of heart 12. In the example shown in FIG. 1, right ventricular (RV) lead 18 extends through one or more veins (not shown), the superior vena cava (not shown), and right atrium 26, and into right ventricle 28. Left ventricular (LV) coronary sinus lead 20 extends through one or more veins, the vena cava, right atrium 26, and into the coronary sinus 30 to a region adjacent to the free wall of the surface of the left ventricle 32 of heart 12. Right atrial (RA) lead 22 extends through one or more veins and the vena cava, and into the right atrium 26 of heart 12.

IMD 16 may sense electrical signals attendant to the depolarization and repolarization of heart 12 via electrodes (not shown in FIG. 1) coupled to at least one of the leads 18, 20, 22. The configurations of electrodes used by IMD 16 for sensing may be unipolar (e.g., using a lead electrode and a can electrode) or bipolar (e.g., using two lead electrodes). IMD 16 may collect, for example, cardiac signals in the form of an electrogram (EGM), which may be used to determine a heart rate interval (e.g., R−R interval), presence or absence of heart rate variability (HRV), presence or absence of non-sustained ventricular tachycardia (NSVT), or the like.

One or more of leads 18, 20, 22 may also carry a pressure sensor 34. In the example illustrated in FIG. 1, pressure sensor 34 is attached adjacent a distal end of lead 18 and positioned in right ventricle 28. Pressure sensor 34 may respond to an absolute pressure inside right ventricle 28, and may be, for example, a capacitive sensor, piezoelectric sensor, mechanical sensor, fiber optic sensor, or the like. In other examples, pressure sensor 34 may be positioned within other regions of heart 12 and may monitor pressure within one or more of the other regions of heart 12, or may be positioned elsewhere within or proximate to the cardiovascular system of patient 14 to monitor cardiovascular pressure associated with mechanical contraction of the heart 12. For example, pressure sensor 34 may be positioned within right atrium 26, left atrium 30, left ventricle 32, or a vein or artery.

Placement of pressure sensor 34 in right ventricle 28 may enable measurement of a variety of hemodynamic parameters by IMD 16. For example, pressure sensor 34 may be used to detect right ventricular (RV) systolic and diastolic pressures (RVSP and RVDP), estimated pulmonary artery diastolic pressure (EPAD), and pressure changes with respect to time (dP/dt). Some parameters may be derived from other parameters, rather than being directly detected by pressure sensor 34. For example, the EPAD parameter may be derived from RV pressure at the moment of pulmonary valve opening.

Pressure sensor 34 in the example of FIG. 1 may be used to detect pressure data relating to right ventricular (RV) pressure. In other examples, however, it is contemplated that any type of sensor could be used, such as a self-contained implantable pressure sensor or a flow sensor in the venous or arterial system. Further, the blood pressure can be detected in other locations of patient 14, including other chambers of heart 12. For example, pressure sensor 34 may be positioned to detect, for example, a left ventricular systolic pressure (LVSP), a left ventricular diastolic pressure (LVDP), a left ventricular pulse pressure (LVPP), a left atrial pressure (LAP), or a right atrial pressure (RAP), in various example implementations.

In some examples, programmer 24 may be a handheld computing device or a computer workstation. Programmer 24 may include a user interface that receives input from a user. The user interface may include, for example, a keypad and a display, which may for example, be a cathode ray tube (CRT) display, a liquid crystal display (LCD) or light emitting diode (LED) display. The keypad may take the form of an alphanumeric keypad or a reduced set of keys associated with particular functions. Programmer 24 can additionally or alternatively include a peripheral pointing device, such as a mouse, via which a user may interact with the user interface. In some examples, a display of programmer 24 may include a touch screen display, and a user may interact with programmer 24 via the display.

A user, such as patient 14, a physician, technician, or other clinician, may interact with programmer 24 to communicate with IMD 16. For example, the user may interact with programmer 24 to retrieve physiological or diagnostic information from IMD 16. A user may also interact with programmer 24 to program IMD 16, e.g., to select values for operational parameters of the IMD 16.

For example, a user such as a clinician may use programmer 24 to retrieve information from IMD 16 regarding the rhythm of heart 12 (e.g., R−R intervals), trends therein over time, or response of the rhythm of heart 12 to changes in blood pressure, referred to as BRS. As another example, the user may use programmer 24 to retrieve information from IMD 16 regarding other sensed physiological parameters of patient 14, such as intracardiac or intravascular pressure, activity, posture, respiration, or thoracic impedance. As a further example, the user may use programmer 24 to retrieve information from IMD 16 regarding the performance or integrity of IMD 16 or other components of system 10, such as leads 18, 20, and 22, or a power source of IMD 16. In some examples, programmer 24 may also receive alerts from IMD 16, such as an alert generated in response to a risk stratification indicator when a BRS measurement obtained by IMD 16 indicates increased risk to patient 14.

IMD 16 and programmer 24 may communicate via wireless communication using any techniques known in the art. Examples of communication techniques may include, for example, low frequency or radiofrequency (RF) telemetry, but other techniques are also contemplated. In some examples, programmer 24 may include a programming head that may be placed proximate to the body of patient 14 near the IMD 16 implant site in order to improve the quality or security of communication between IMD 16 and programmer 24.

IMD 16 may obtain a BRS measurement of patient 14 by detecting the rate of heart 12 via combinations of electrodes carried by at least one of leads 18, 20, 22, and a blood pressure of patient 14 via pressure sensor 34. BRS is a measure of the ability of heart 12 to react to changes in blood pressure by changing heart rate. Typically, a decrease in heart rate (i.e., increase in RR interval) is associated with an increase in blood pressure up to a certain point where the signals start to deviate. Similarly, heart rate typically increases as pressure decreases. However, in patients with heart failure, blood pressure and heart rate do not track together well. As the heart failure of patient 14 worsens, the tracking of blood pressure and heart rate also worsens. In some examples, BRS may be measured as the slope of a linear regression line that fits the increase in blood pressure with an increase in RR intervals. A low BRS measurement may reflect this reduced tracking of blood pressure and heart rate.

Because BRS measurements correlate to heart failure of patient 14, IMD 16 or programmer 24 may utilize the BRS measurement to generate a risk stratification indicator for patient 14. The risk stratification indicator may indicate the risk of having future cardiac arrhythmias or cardiac mortality for patient 14. In some cases, the risk stratification indicator may serve to classify the patient 14 among two or more different risk strata, i.e., cardiac mortality or cardiac arrhythmia vulnerability risk categories, each of which may be correlated with candidacy for IMD implantation.

For example, as will be described in further detail below, IMD 16 or programmer 24 may automatically generate an implantation indicator based on generation of the risk stratification indicator, or based on a value of the risk stratification indicator. In either case, the risk stratification indicator may provide an indication that patient 14 is a candidate for implantation of an implantable therapy device, such as a cardiac pacemaker, an implantable cardioverter-defibrillator (ICD), a cardiac resynchronization therapy (CRT) pacing device, or a drug delivery device. A clinician may act on the implantation indicator as a recommendation, and elect to proceed with implantation of an IMD in patient 14. In general, a patient may be considered a candidate for implantation of the device if the risk stratification indicator indicates that the patient is critically in need of the device or would benefit from the device in order to reduce cardiac arrhythmia or cardiac mortality risk.

Alternatively, instead of generating an automatic implantation indicator, a clinician may review the risk stratification indicator and use the risk stratification indicator to determine whether patient 14 is a candidate for implantation of one of the implantable therapy devices, or whether to prescribe a drug to the patient 14. As a further alternative, the risk stratification indicator may be used by IMD 16, programmer 24, or a clinician to prescribe adjustment of an existing cardiac therapy, such as one or more parameters associated with cardiac electrical stimulation therapy or dosages associated with one or more drugs. In other examples, the risk stratification indicator may be used by an implantable drug delivery device to prescribe adjustment of an existing drug delivery therapy. IMD 16 or programmer 24 may also generate an alert to a user, such as patient 14 or a clinician, based on the risk stratification indicator. The alert may indicate that the condition of patient 14 is changing or has changed.

In some examples, the risk stratification indicator may comprise a binary output, classifying the patient into one of two cardiac arrhythmia or cardiac mortality risk categories, such as risk or no risk, or high risk or low risk, or one of a plurality of risk levels corresponding to three or more cardiac arrhythmia or cardiac mortality risk categories (e.g., low risk, medium risk, high risk or very low risk, low risk, medium risk, high risk or very high risk). In turn, IMD 16 or programmer 24 may automatically generate a binary implant indicator such as implant or no implant, or a range of implant indicators such as implant critically needed, patient would benefit from implant, implant not needed but may be beneficial, implant not needed but optional, or no implant benefit likely. Hence, IMD 16 or programmer 24 may generate different implant indications for presentation to a clinician or other user for different, corresponding values of the risk stratification indicator.

IMD 16 includes leads 18, 20, 22, which carry pressure sensor 34 and electrodes that measure cardiac signals, and may thus obtain continuous or chronic BRS measurements. This may provide the ability to monitor a condition of patient 14 in between clinical visits, and may also enable IMD 16, programmer 24, or another computing device to produce trends of the BRS measurements over time, which may indicate a change in the condition of patient 14, and a progression of heart failure in the patient.

In some examples, IMD 16 may utilize the cardiac signals detected by electrodes carried by one or more of leads 18, 20, 22 to determine other cardiac measurements, such as, for example, a HRV measurement or a NSVT indicator. IMD 16 or programmer 24 may then generate the risk stratification indicator based on the BRS measurement alone, or the BRS measurement in combination with one or both of the HRV measurement and the NSVT indicator, as will be described in further detail below. In other examples, IMD 16 or programmer 24 may generate the risk stratification indicator based on the BRS measurement in combination with one or more of the HRV measurement, an ejection fraction measurement, the NSVT indicator, an age of patient 14, gender of patient 14, history of heart failure or cardiac disease of patient 14, or the like.

FIG. 2 is a conceptual diagram illustrating an exemplary therapy system 38, including programmer 24, an IMD 40 and leads 42, 44, 46. Leads 42, 44, 46 may be electrically coupled to an electrical stimulation generator, a sensing module, or other modules of IMD 40 via connector block 48. In some examples, proximal ends of leads 42, 44, 46 may include electrical contacts that electrically couple to respective electrical contacts within connector block 48. In addition, in some examples, leads 42, 44, 46 may be mechanically coupled to connector block 48 with the aid of set screws, connection pins or another suitable mechanical coupling mechanism.

Each of the leads 42, 44, 46 includes an elongated insulative lead body, which may carry a number of concentric coiled conductors separated from one another by tubular insulative sheaths. In the illustrated example, a pressure sensor 34 and bipolar electrodes 52 and 54 are located proximate to a distal end of lead 42. In addition, bipolar electrodes 56 and 58 are located proximate to a distal end of lead 44 and bipolar electrodes 60 and 62 are located proximate to a distal end of lead 46. In FIG. 2, pressure sensor 34 is again disposed in right ventricle 28 for purposes of illustration. Pressure sensor 34 may respond to an absolute pressure inside right ventricle 28, and may be, for example, a capacitive sensor, piezoelectric sensor, mechanical sensor, fiber optic sensor, or the like. In other examples, pressure sensor 34 may be positioned within other regions of heart 12 and may monitor pressure within one or more of the other regions of heart 12, or may be positioned elsewhere within or proximate to the cardiovascular system of patient 14 to monitor cardiovascular pressure associated with mechanical contraction of the heart.

Electrodes 52, 56, and 60 may take the form of ring electrodes, and electrodes 54, 58 and 62 may take the form of extendable helix tip electrodes mounted retractably within insulative electrode heads 64, 66 and 68, respectively. Each of the electrodes 52, 54, 56, 58, 60 and 62 may be electrically coupled to a respective one of the coiled conductors within the lead body of its associated lead 42, 44, 46, and thereby coupled to respective ones of the electrical contacts on the proximal end of leads 42, 44 and 46.

Electrodes 52, 54, 56, 58, 60 and 62 may sense electrical cardiac signals attendant to the depolarization and repolarization of heart 12. The cardiac signals are conducted to IMD 40 via the respective leads 42, 44, 46. IMD 40 also delivers pacing pulses via electrodes 52, 54, 56, 58, 60 and 62 to cause depolarization of cardiac tissue of heart 12. In some examples, as illustrated in FIG. 2, IMD 40 includes one or more housing electrodes, such as housing electrode 70, which may be formed integrally with an outer surface of hermetically-sealed housing 72 of IMD 40 or otherwise coupled to housing 72. In some examples, housing electrode 70 is defined by an uninsulated portion of an outward facing portion of housing 72 of IMD 40. Other divisions between insulated and uninsulated portions of housing 72 may be employed to define two or more housing electrodes. In some examples, housing electrode 70 comprises substantially all of housing 72. Any of the electrodes 52, 54, 56, 58, 60 and 62 may be used for unipolar sensing or pacing in combination with housing electrode 70. As described in further detail with reference to FIG. 4, housing 72 may enclose a stimulation generator that generates cardiac pacing pulses or waveforms and defibrillation or cardioversion shocks, as well as a cardiac sensing module for monitoring the rhythm and other attributes of heart 12.

Leads 42, 44, and 46 also include elongated electrodes 74, 76, 78, respectively, which may take the form of a coil. IMD 40 may deliver cardioversion and/or defibrillation shocks to heart 12 via any combination of elongated electrodes 74, 76, 78, and housing electrode 70. Electrodes 74, 76, 78 may be fabricated from any suitable electrically conductive material, including, but not limited to, platinum, a platinum alloy or other materials known to be usable in implantable defibrillation electrodes.

Pressure sensor 34 may be coupled to one or more elongated, coiled conductors within lead 42. In FIG. 2, pressure sensor 34 is located more distally on lead 18 than elongated electrode 74. In other examples, pressure sensor 34 may be positioned more proximally than elongated electrode 74, rather than distal to electrode 74. Further, pressure sensor 34 may be coupled to another one of the leads 44, 46 in other examples, or to a lead other than leads 42, 44, 46 carrying stimulation and sense electrodes. In addition, in some examples, pressure sensor 34 may be self-contained device that is implanted within heart 12, such as within the septum separating right ventricle 28 from left ventricle 32, or the septum separating right atrium 26 from left atrium 33. In such an example, pressure sensor 34 may wirelessly communicate with IMD 40.

Similar to IMD 16, IMD 40 may obtain a BRS measurement by detecting the rate (e.g., R−R interval) of heart 12 and blood pressure of patient 14. IMD 40 or programmer 24 may use the BRS measurements to generate a risk stratification indicator. Again, the risk stratification indicator may indicate the risk of cardiac arrhythmia or cardiac mortality to patient 14, and may categorize the patient into one of two or more cardiac arrhythmia or cardiac mortality risk categories (e.g., low risk, medium risk, high risk). The risk stratification indicator may be presented to a user such as a clinician via programmer 24 or another computing device to permit the clinician to quickly ascertain the cardiac arrhythmia or cardiac mortality risk status of the patient, and consider an appropriate course of action, such as implantation of a cardiac electrical stimulation therapy device.

Based on the risk stratification indicator, in some examples, IMD 40 or programmer 24 may automatically generate an instruction to initiate or modify a therapy program according to which IMD 40 delivers stimulation to heart 12. For example, the IMD 40 or programmer 24 may initiate resetting or suspension of the current therapy program by IMD 40 based on the risk stratification indicator, or may direct IMD 40 to switch to a different therapy program based on the risk stratification indicator. Each therapy program may define a plurality of stimulation parameters, including, for example, stimulation pulse width, stimulation pulse amplitude, stimulation frequency, an electrode configuration and/or polarity, or the like. IMD 40 of programmer 24 may also generate an alert to a user, such as patient 14 or a clinician, based on the risk stratification indicator. The alert may comprise a notification that the condition of patient 14 is changing or has changed. Again, in some examples, the risk stratification indicator may comprise a binary output, such as risk or no risk, or high risk or low risk, or may comprise one of a plurality of risk levels (e.g., very low risk, medium risk, high risk). In this manner, the risk stratification indicator may categorize the patient into one of two or more cardiac arrhythmia or cardiac mortality risk categories for convenient interpretation by a clinician. For example, in contrast to raw BRS values, the risk categories may be expressed textually (e.g., low, medium, high, or mild cardiac arrhythmia, severe cardiac arrhythmia, cardiac mortality) to permit ready interpretation, in a simple numeric format (e.g., 1, 2, 3 or A, B, C), or in a color-coded format (e.g., green, yellow, red).

In some examples, IMD 16 may utilize the cardiac signals detected by electrodes carried by one or more of leads 18, 20, 22 to determine other physiological measurements, such as, for example, an HRV measurement or a NSVT indicator. IMD 16 and/or programmer 24 may then generate the risk stratification indicator based on the BRS measurement and at least one of the HRV measurement and the NSVT indicator. In some examples, IMD 16 or programmer 24 may generate the risk stratification indicator based on the BRS measurement in combination with one or more of the HRV measurement, an ejection fraction measurement, the NSVT indicator, an age of patient 14, gender of patient 14, history of heart failure or cardiac disease of patient 14, or the like.

The configurations of monitoring system 10 illustrated in FIG. 1 and therapy system 38 illustrated in FIG. 2 are merely two examples. In other examples, a monitoring system or therapy system may include epicardial leads and/or patch electrodes instead of or in addition to the transvenous leads 18, 20, 22, 42, 44, 46 illustrated in FIGS. 1 and 2.

In other examples of therapy systems that provide electrical stimulation therapy to heart 12, a therapy system may include any suitable number of leads coupled to IMD 40, and each of the leads may extend to any location within or proximate to heart 12. For example, other examples of therapy systems may include three transvenous leads located as illustrated in FIGS. 1 and 2, and an additional lead located within or proximate to left atrium 33. As another example, other examples of therapy systems may include a single lead that extends from IMD 16 into right atrium 26 or right ventricle 28, or two leads that extend into a respective one of the right ventricle 26 and right atrium 28.

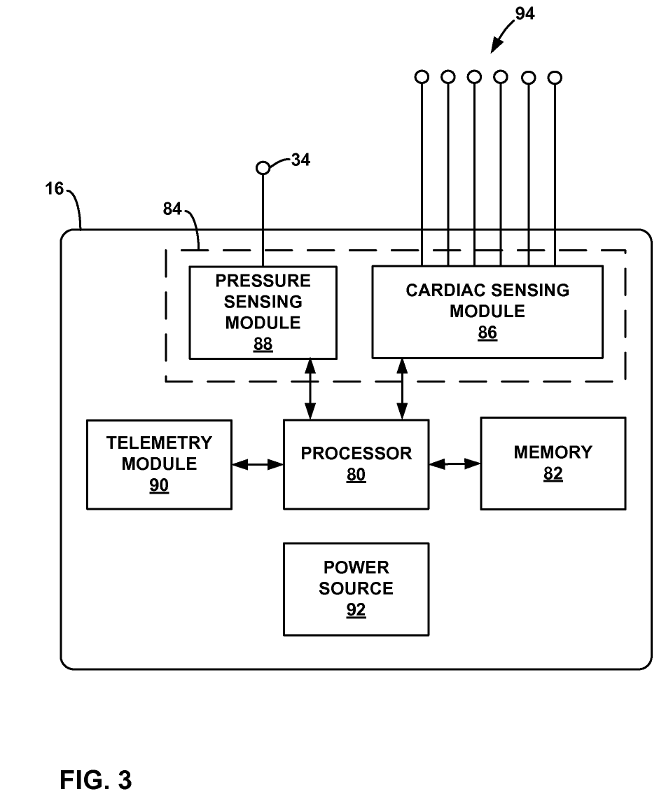

FIG. 3 is a functional block diagram of one example configuration of IMD 16, which includes a processor 80, memory 82, a measurement unit 84, a telemetry module 90, and a power source 92. In the example of FIG. 3, measurement unit 84 includes a cardiac sensing module 86 and a pressure sensing module 88.

Memory 82 includes computer-readable instructions that, when executed by processor 80, cause IMD 16 and processor 80 to perform various functions attributed to IMD 16 and processor 80 herein. Memory 82 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, magneto-resistive random access memory (MRAM), or any other digital media.

Processor 80 may include any one or more of a microprocessor, a controller, a digital signal processor (DSP), an application specific integrated circuit (ASIC), a field-programmable gate array (FPGA), or equivalent discrete or integrated logic circuitry. In some examples, processor 80 may include multiple components, such as any combination of one or more microprocessors, one or more controllers, one or more DSPs, one or more ASICs, or one or more FPGAs, as well as other discrete or integrated logic circuitry. The functions attributed to processor 80 herein may be embodied as software, firmware, hardware or any combination thereof.

Measurement unit 84 may obtain a BRS measurement by detecting one or more physiological parameters of patient 14. For example, in the example illustrated in FIG. 3, measurement unit 84 includes a cardiac sensing module 86, which detects a cardiac signal of heart 12, and a pressure sensing module 88, which detects a blood pressure of patient 14.

Cardiac sensing module 86 may detect cardiac signals via at least one of a plurality of electrodes 94 in order to monitor electrical activity of heart 12, e.g., by constructing an electrogram (EGM) from the cardiac signals. Electrodes 94 may be dedicated sensing electrodes if IMD 16 is configured as a physiological signal monitoring device. Alternatively, electrodes 94 may form dedicated sensing electrodes or combined sensing/stimulation electrodes in examples in which IMD 16 is configured to also deliver electrical stimulation. Cardiac sensing module 86 may also include a switch module to select which of the available electrodes 94 are used to sense the cardiac activity. In some examples, processor 80 may select the electrodes 94 that function as sense electrodes via the switch module within cardiac sensing module 86, e.g., by providing signals via a data/address bus. In some examples, cardiac sensing module 86 includes one or more sensing channels, each of which may comprises an amplifier. In response to the signals from processor 80, the switch module within cardiac sensing module 86 may couple the outputs from the selected electrodes to one of the sensing channels.

In some examples, one channel of cardiac sensing module 86 may include an amplifier that receives signals from electrodes 94, which may be used for sensing R-waves in right ventricle 28 of heart 12. Another channel may include another amplifier that receives signals from electrodes (not shown) that are used for R-wave sensing proximate to left ventricle 32 of heart 12. In some examples, the amplifiers may each take the form of an automatic gain controlled amplifier that provides an adjustable sensing threshold as a function of the measured R-wave amplitude of rhythm of heart 12. The amplifiers and corresponding sensing channels of sensing module 86 detect R waves for use in establishing an R−R interval for the heart of patient 14. The R−R interval indicates the time between successive ventricular depolarizations, e.g., in the right ventricle or in the left ventricle. The R−R interval indicates the cardiac cycle length, which may be converted to express heart rate in terms of beats per minute.

In some examples, cardiac sensing module 86 includes a channel that comprises an amplifier with a relatively wider pass band than the R-wave sensing amplifier(s). Signals from the selected sensing electrodes that are selected for coupling to this wide-band amplifier may be provided to a multiplexer, and thereafter converted to multi-bit digital signals by an analog-to-digital converter for storage in memory 82 as an EGM. In some examples, the storage of such EGMs in memory 82 may be under the control of a direct memory access circuit. Processor 80 may employ digital signal analysis techniques to characterize the digitized signals stored in memory 82 to detect and classify the rhythm of heart 12 from the cardiac signals. Processor 80 may detect and classify the rhythm of heart 12 by employing any of the numerous signal processing methodologies known in the art.

For example, processor 80 may determine the R−R interval from the cardiac signal obtained from the wide-band amplifier channel or the R wave detections provided by the R wave amplifier channels. Again, the R−R interval is the length of time between consecutive R-waves, i.e., ventricular depolarizations, and represents the cardiac cycle length. In some examples processor 80 may determine an R−R interval for each of a plurality of consecutive R-waves, and may store the R−R intervals in memory 82. Processor 80 may use one or more of the determined R−R intervals in determining the BRS measurement, as will be described in further detail below.

Pressure sensing module 88 may receive pressure signals from pressure sensor 34. The pressure signals are a function of the fluid pressure at the site where pressure sensor 34 is disposed. In the example shown in FIG. 1, pressure sensor 34 is disposed in right ventricle 28 of heart 12. In other examples, pressure sensor 34 may be disposed in other chambers of heart 12, such as left ventricle 32, or may be disposed in an artery or vein of patient 14. Pressure sensing module 88 may receive, monitor, and analyze the pressure signals, as will be described in more detail below. An example of a suitable pressure sensing module 88 includes the Chronicle Implantable Hemodynamic Monitor manufactured by Medtronic, Inc. of Minneapolis, Minn.

Pressure sensing module 88, or, alternatively, processor 80, may measure, observe, or derive different pressure characteristics from the signals generated by pressure sensor 34. For instance, in examples in which pressure sensor 34 generates a signal indicative of the pressure within right ventricle 28, pressure sensing module 88 may measure the RVSP by observing a peak pressure in right ventricle 28. In addition, pressure sensing module 88 may measure the RVDP by observing the pre-systolic low pressure in right ventricle 28. Pulse pressure may be the difference between the RVSP and the RVDP.

Another pressure characteristic that pressure sensing module 88 may measure is the right ventricular mean pressure (RVMP), which is the mean pressure in right ventricle 28 during a cardiac cycle. A cardiac cycle (or “heart cycle”) typically includes a P-wave, Q-wave, an R-wave, and an S-wave (forming a QRS complex), and a T-wave. Pressure sensing monitor 90 may also monitor EPAD, which is another pressure characteristic that may be indicative of activity within heart 12. EPAD reflects the pulmonary capillary wedge pressure, which reflects the average pressure in left atrium 33 over a cardiac cycle, which may also be referred to as the mean left atrial pressure. EPAD may also reflect the filling pressure in left ventricle 32 during diastole, also called the left ventricular end diastolic pressure.

Example techniques for measuring EPAD are described in U.S. Pat. No. 7,058,450 to Struble et al., entitled, “ORGANIZING DATA ACCORDING TO CARDIAC RHYTHM TYPE,” which issued on Jun. 6, 2006 and is incorporated herein by reference in its entirety. In various examples, pressure may be measured in other chambers of heart 12, or other locations within the cardiovascular system of patient 14, such as within a pulmonary artery. As other examples, pressure sensor 34 may be positioned to detect a left ventricular systolic pressure (LVSP), a left ventricular diastolic pressure (LVDP), a left ventricular pulse pressure (LVPP), a left atrial pressure (LAP), or a right atrial pressure (RAP).

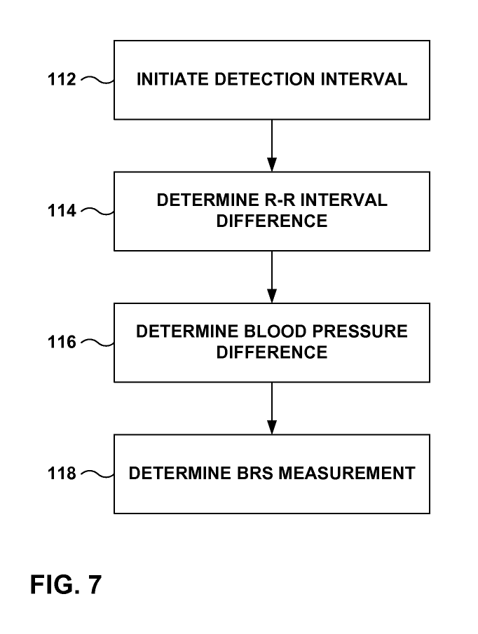

Processor 80 may determine the BRS measurement based on the blood pressure signal from pressure sensor 34 or a pressure signal output by pressure sensing module 88 and cardiac signals from sensing module 86. For example, processor 80 may determine a difference in blood pressure (ΔBP) based on the blood pressure signal and a difference in R−R interval (ΔR−R) based on the cardiac signals, as described in further detail with reference to FIG. 7. Processor 80 then may compute the BRS measurement as:

$$B R S = \frac{Δ R - R ( ms )}{Δ B P ( mm Hg )}$$

where ΔR−R is the difference in R−R interval measured in milliseconds (ms) and ΔBP is the difference in blood pressure measured in mm Hg. The difference ΔR−R represents the difference between the maximum R−R interval and the minimum R−R interval during a given time period during which the R−R interval is monitored. The difference ΔBP represents the difference between the maximum blood pressure and the minimum blood pressure for the same period of time. For example, the difference ΔBP may be a right ventricular pressure difference.

In other examples, processor 80 may compute the BRS measurement by performing linear regression analysis of a plurality of R−R interval and blood pressure pairs (e.g., a blood pressure measurement collected during the same cardiac cycle as the respective R−R interval measurement). For example, processor 80 may determine R−R intervals and blood pressures for a plurality, e.g., five, consecutive cardiac cycles, and may perform linear regression analysis on these five R−R interval and blood pressure pairs. The BRS measurement is the slope of the linear regression line. In this manner, the computation of BRS may use several R−R intervals and corresponding blood pressure points, e.g., over 5 consecutive beats as mentioned above, and calculate a linear regression line through the points. The slope of this line is the BRS value.

Processor 80 also may compute an average BRS measurement from a plurality of BRS measurements, and may store this average in memory 82 as the BRS measurement. For example, processor 80 may average five consecutive BRS measurements into a single average BRS measurement and may store this average BRS measurement in memory 82. In other words, BRS may be computed as the average of n (e.g., n=5) consecutive slope measurements of the ratio of R−R interval differences to blood pressure differences.

Processor 80 also may determine an HRV measurement from the cardiac signal. The HRV measurement is a measure of variation among R−R intervals, and may be determined as a standard deviation of the R−R interval. In order to determine the HRV measurement, processor 80 may determine and store a plurality of R−R intervals obtained over a period of time in memory 82. In some examples, the plurality of R−R intervals comprises a plurality of consecutive R−R intervals, such as, for example, about 15 consecutive R−R intervals. Processor 80 may determine the HRV measurement as the standard deviation of the plurality of R−R intervals. The value of the HRV measurement may indicate an increased risk of cardiac arrhythmia or cardiac mortality for patients with previous heart problems. For example, an HRV measurement of less than about 70 ms may indicate increased risk to patient 14.

Processor 80 may also determine a non-sustained ventricular tachycardia (NSVT) indicator from the cardiac signal or from the R-wave events indicated by the R wave amplifier channels. Ventricular tachycardia refers to a heart rate in excess of 100 beats per minute (bpm). A NSVT, then, refers to a heart rate that exceeds 100 bpm, but lasts less than about 30 seconds and ceases without intervention. In order for a NSVT to be present, a heart rate in excess of, for example, 100 bpm must be present for at least three consecutive heart beats. In other examples, NSVT may be defined as a heart rate that exceeds 120 bpm for three consecutive heart beats, but lasts less than about 30 seconds and ceases without intervention.

The NSVT indicator may be determined from the cardiac signal by determining the R−R interval and comparing the R−R interval to a threshold value. For example, an R−R interval of about 600 milliseconds (ms) corresponds to a heart rate of about 100 bpm. In some examples, the threshold R−R interval may be set at about 600 ms and three consecutive R−R intervals of less than 600 ms may indicate a NSVT. In other examples, the threshold R−R interval may be set to a value less than 600 ms, such as, for example, 300 ms. The threshold R−R interval may be determined by a clinician and stored in memory 82 of IMD 16. The presence of NSVT may indicate an increased risk of mortality for patients with previous heart problems.

Processor 80 may generate a risk stratification indicator based on the BRS measurement alone, or based on the BRS measurement in combination with at least one of the HRV measurement and the NSVT indicator. For example, processor 80 may generate the risk stratification indicator by comparing the BRS measurement to a threshold value (for example, 3 milliseconds/millimeter Hg), as will be described in further detail below. Processor 80 may generate the risk stratification indicator when the BRS measurement is below the threshold value, which indicates a depressed ability of heart 12 to respond to changes in blood pressure. In some examples, IMD 16 or programmer 24 may generate the risk stratification indicator based on the BRS measurement in combination with one or more of the HRV measurement, an ejection fraction measurement, the NSVT indicator, an age of patient 14, gender of patient 14, history of heart failure or cardiac disease of patient 14, or the like.

Processor 80 may then generate, for example, an alert to a user, such as patient 14 or a clinician, based on the risk stratification indicator. In other examples, processor 80 may generate based on the risk stratification indicator an indicator that patient 14 is a candidate for an IMD that provides therapy, such as stimulation therapy or drug delivery, or an indicator that presently prescribed therapy, such as stimulation therapy or drug delivery, should be adjusted. The risk stratification indicator may comprise a binary output (e.g., risk or no risk or high risk or low risk), or one of a plurality of risk levels (e.g., very low risk, medium risk, high risk), in which case multiple thresholds for each category may be utilized as described below.

Telemetry module 90 includes any suitable hardware, firmware, software or any combination thereof for communicating with another device, such as programmer 24 (FIG. 1). Under the control of processor 80, telemetry module 90 may receive downlink telemetry from and send uplink telemetry to programmer 24 with the aid of an antenna, which may be internal and/or external. Processor 80 may provide the data to be uplinked to programmer 24 and the control signals for the telemetry circuit within telemetry module 86, e.g., via an address/data bus. In some examples, telemetry module 90 may provide received data to processor 80 via a multiplexer.

In some examples, processor 80 may transmit atrial and/or ventricular cardiac signals (e.g., EGM signals) produced by atrial and/or ventricular sense amplifier circuits within cardiac sensing module 86 and blood pressure signals produced by pressure sensing module 88 to programmer 24. Programmer 24 may interrogate IMD 16 to receive the cardiac and blood pressure signals. Processor 80 may store the cardiac and blood pressure signals within memory 82, and retrieve stored cardiac and blood pressure signals from memory 82. Processor 80 may also generate and store marker channel codes indicative of different cardiac episodes that cardiac sensing module 86 or processor 80 detects, and transmit the marker codes to programmer 24. An example pacemaker with marker-channel capability is described in U.S. Pat. No. 4,374,382 to Markowitz, entitled, “MARKER CHANNEL TELEMETRY SYSTEM FOR A MEDICAL DEVICE,” which issued on Feb. 15, 1983 and is incorporated herein by reference in its entirety.

In other examples, processor 80 may transmit parametric data derived from atrial and/or ventricular cardiac signals produced by cardiac sensing module 86 and blood pressure signals produced by pressure sensing module 88 to programmer 24. In particular, processor 80 may transmit R−R interval data, blood pressure data, RR difference data, and/or blood pressure difference data, such as right ventricular blood pressure difference data in some examples. Hence, in various implementations, processor 80 may generate BRS, HRV, and NVST indicators and a risk stratification indicator within IMD 16, or transmit raw data, processed data or parametric data to programmer 24 for generation of one or more of the BRS, HRV, NVST, and risk stratification indicator. Parametric data may include particular intervals or values, or information such as marker channel data useful in determining intervals or values.

The various components of IMD 16 may be coupled to power source 92, which may include a rechargeable or non-rechargeable battery and suitable power supply circuitry. A non-rechargeable battery may be selected to last for several years, while a rechargeable battery may be inductively charged from an external device, e.g., on a daily or weekly basis.

Although FIG. 3 illustrates cardiac sensing module 86 and pressure sensing module 88 as separate components from processor 80, in other examples, processor 80 may include some of the functionality attributed to cardiac sensing module 86 and pressure sensing module 88 in this disclosure. For example, cardiac sensing module 86 and/or pressure sensing module 88 shown in FIG. 3 may include software executed by processor 80. If cardiac sensing module 86 or pressure sensing module 88 includes firmware or hardware, cardiac sensing module 86 or pressure sensing module 88 may be a separate one of the one or more processors 80 or may be a part of a multifunction processor. As previously described, processor 80 may comprise one or more processors.

Further, in other examples of monitoring system 10 or therapy system 38, cardiac sensing module 86 or pressure sensing module 88 may be separate from IMD 16, 40. That is, although cardiac sensing module 86 and pressure sensing module 88 are shown in FIG. 3 to be incorporated within or coupled to a housing of IMD 16 along with other components such as processor 80, in other examples, cardiac sensing module 86 or pressure sensing module 88 may be enclosed in a separate housing. A stand-alone cardiac sensing module or pressure sensing module that is enclosed in a separate housing from the housing of IMD 16 may be mechanically coupled to IMD 16 or may be mechanically decoupled from IMD 16. For example, in some examples, pressure sensing module 88 and pressure sensor 34 may be implanted within patient 14 at a separate location from IMD 16 and leads 18, 20, 22. Cardiac sensing module 86 or pressure sensing module 88 may communicate with IMD 16 via a wired connection or via wireless communication techniques, such as RF telemetry.

FIG. 4 is a functional block diagram of one example configuration of IMD 40, which includes processor 80, memory 82, measurement unit 84 including sensing module 86 and pressure sensing module 88, telemetry module 90, power source 92, and a stimulation generator 98. In addition to the functions of processor 80 described above with respect to FIG. 3, processor 80 in FIG. 4 also may control stimulation generator 98 to deliver stimulation therapy to heart 12 according to a selected on or more therapy programs, which may be stored in memory 82. Specifically, processor 80 may control stimulation generator 96 to deliver electrical waveforms, pulses, or shocks with the amplitudes, pulse widths, frequency, or electrode polarities specified by the selected on or more therapy programs.

Stimulation generator 98 is electrically coupled to electrodes 52, 54, 56, 58, 60, 62, 70, 74, 76, 78, e.g., via conductors of the respective lead 42, 44, 46, or, in the case of housing electrode 70, via an electrical conductor disposed within housing 72 of IMD 40. Stimulation generator 98 is configured to generate and deliver electrical stimulation therapy to heart 12. For example, stimulation generator 74 may deliver defibrillation shocks to heart 12 via at least two electrodes 70, 74, 76, 78. Stimulation generator 98 may deliver pacing pulses or waveforms via ring electrodes 52, 56, 60 coupled to leads 42, 44, and 46, respectively, and/or helical electrodes 54, 58, 62 of leads 42, 44, and 46, respectively. In some examples, stimulation generator 98 delivers pacing, cardioversion, or defibrillation stimulation in the form of electrical pulses. In other examples, stimulation generator 98 may deliver one or more of these types of stimulation in the form of other signals, such as sine waves, square waves, or other substantially continuous time signals.

Stimulation generator 98 may include a switch module and processor 80 may use the switch module to select, e.g., via a data/address bus, which of the available electrodes are used to deliver defibrillation pulses or pacing pulses. The switch module may include a switch array, switch matrix, multiplexer, or any other type of switching device suitable to selectively couple stimulation energy to selected electrodes.

Processor 80 may include a pacer timing and control module, which may be embodied as hardware, firmware, software, or any combination thereof The pacer timing and control module may comprise a dedicated hardware circuit, such as an ASIC, separate from other components of processor 80, such as a microprocessor, or a software module executed by a component of processor 80, which may be a microprocessor or ASIC. The pacer timing and control module may include programmable counters which control the basic time intervals associated with DDD, VVI, DVI, VDD, AAI, DDI, DDDR, VVIR, DVIR, VDDR, AAIR, DDIR, and other modes of single and dual chamber pacing. In the aforementioned pacing modes, “D” may indicate dual chamber, “V” may indicate a ventricle, “I” may indicate inhibited pacing (e.g., no pacing), and “A” may indicate an atrium. The first letter in the pacing mode may indicate the chamber that is paced, the second letter may indicate the chamber in which an electrical signal is sensed, and the third letter may indicate the chamber in which the response to sensing is provided.

Intervals defined by the pacer timing and control module within processor 80 may include atrial and ventricular pacing escape intervals, refractory periods during which sensed P-waves and R-waves are ineffective to restart timing of the escape intervals, and the pulse widths of the pacing pulses. As another example, the pace timing and control module may define a blanking period, and provide signals from sensing module 86 to blank one or more channels, e.g., amplifiers, for a period during and after delivery of electrical stimulation to heart 12. The durations of these intervals may be determined by processor 80 in response to stored data in memory 82. The pacer timing and control module of processor 80 may also determine the amplitude of the cardiac pacing pulses or waveforms.

During pacing, escape interval counters within the pacer timing/control module of processor 80 may be reset upon sensing of R-waves and P-waves. The count at the time a ventricular escape interval is reset indicates the pertinent R−R interval at that time. Stimulation generator 98 may include pacer output circuits that are coupled, e.g., selectively by a switching module, to any combination of electrodes 52, 54, 56, 58, 60, 62, 70, 74, 78 appropriate for delivery of a bipolar or unipolar pacing pulse to one of the chambers of heart 12. Processor 80 may reset the escape interval counters upon the generation of pacing pulses by stimulation generator 98, and thereby control the basic timing of cardiac pacing functions, including anti-tachyarrhythmia pacing.

When IMD 40 is configured to generate and deliver defibrillation shocks to heart 12, stimulation generator 98 may include a high voltage charge circuit and a high voltage output circuit. In the event that generation of a cardioversion or defibrillation shock is required, processor 80 may employ the escape interval counter to control timing of such cardioversion and defibrillation shocks, as well as associated refractory periods. In response to the detection of atrial or ventricular fibrillation of tachyarrhythmia requiring a cardioversion pulse, processor 80 may activate a cardioversion/defibrillation control module, which may, like pacer timing and control module, be hardware component of processor 80 and/or a firmware or software module executed by one or more hardware components of processor 80. The cardioversion/defibrillation control module may initiate charging of the high voltage capacitors of the high voltage charge circuit of stimulation generator 98 under control of a high voltage charging control line.

Processor 80 may monitor the voltage on the high voltage capacitor, e.g., via a voltage charging and potential (VCAP) line. In response to the voltage on the high voltage capacitor reaching a predetermined value set by processor 80, processor 80 may generate a logic signal that terminates charging. Thereafter, timing of the delivery of the defibrillation or cardioversion pulse by stimulation generator 98 is controlled by the cardioversion/ defibrillation control module of processor 80. Following delivery of the fibrillation or tachycardia therapy, processor 80 may return stimulation generator 98 to a cardiac pacing function and await the next successive interrupt due to pacing or the occurrence of a sensed atrial or ventricular depolarization.

Stimulation generator 98 may deliver cardioversion or defibrillation pulses with the aid of an output circuit that determines whether a monophasic or biphasic pulse is delivered, whether housing electrode 70 serves as cathode or anode, and which electrodes are involved in delivery of the cardioversion of defibrillation pulses. Such functionality may be provided by one or more switches or a switching module of stimulation generator 98.

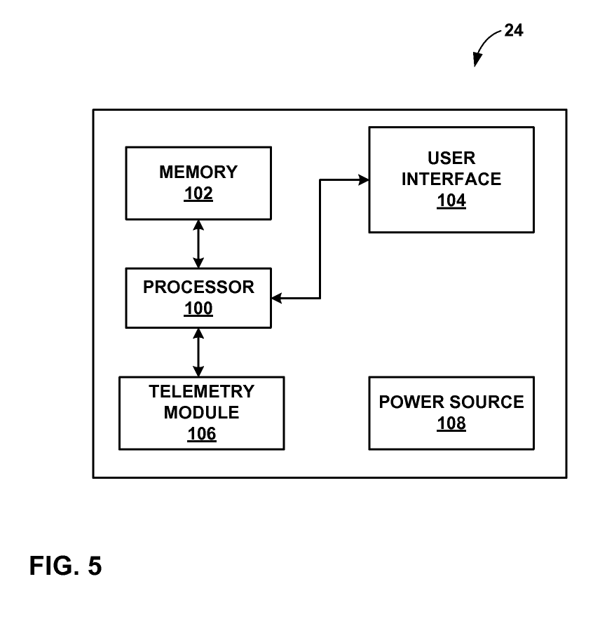

In some examples, processor 80 and/or stimulation generator 98 may be responsive to risk stratification indicator generated by processor 80 or processor 100 of programmer 24 (FIG. 5). In some instances, processor 80 or processor 100 may generate an instruction to initiate or modify a therapy program according to which IMD 40 delivers stimulation to heart 12 based on the risk stratification indicator. For example, the instruction may initiate resetting or suspension of the current therapy program by IMD 40, or may initiate IMD 40 to switch to a different therapy program. Each therapy program may define a plurality of stimulation parameters, including, for example, stimulation pulse width, stimulation pulse amplitude, stimulation frequency, an electrode configuration and/or polarity, or the like. In response to the instruction, processor 80 may control stimulation generator 98 to initiate delivery of stimulation therapy, reset stimulation therapy, cease delivery of stimulation therapy, change one or more therapy program parameters according to which stimulation generator 98 delivers therapy, or otherwise modify stimulation therapy delivered by stimulation generator 98. For example, therapy may be modified to better address a worsening or lessening heart failure condition of patient 14.

FIG. 5 is a functional block diagram of an example programmer 24. As shown in FIG. 5, programmer 24 includes processor 100, memory 102, user interface 104, telemetry module 106, and power source 108. Programmer 24 may be a dedicated hardware device with dedicated software for programming of IMD 16. Alternatively, programmer 24 may be an off-the-shelf computing device running an application that enables programmer 24 to program IMD 16.

A user such as a clinician may use programmer 24 to select therapy programs (e.g., sets of stimulation parameters), generate new therapy programs, modify therapy programs through individual or global adjustments or transmit the new programs to a medical device, such as IMD 40 (FIGS. 2 and 4). The user may also use programmer 24 to program or modify parameters related to the determination of a risk stratification indicator, such as, for example, threshold values to which the BRS measurement, HRV measurement, or NSVT indicator are compared. In some examples, the user also may utilize programmer 24 to modify the frequency or length of detection intervals, the particular perturbation that initiates a detection interval, or the like. The user may interact with programmer 24 via user interface 104, which may include a display to present graphical user interface to a user, and a keypad or another mechanism for receiving input from a user.

The user also may use programmer 24 to retrieve data stored in memory 82 of IMD 16, 40, such as, for example, physiological parameters sensed by sensors communicatively coupled to IMD 16, 40. The physiological parameters may be used by programmer 24 to compute a risk stratification indicator or other related indicators such as BRS, HRV or NSVT. The user further may use programmer 24 to retrieve a risk stratification indicator stored in memory 82 or an implantation indicator stored in memory 82, if computed within IMD 16, or other measurements or indicators related to the computation of the risk stratification indicator (e.g., BRS and HRV measurement, NSVT indicator), if computed within IMD 16. Hence, the BRS, HRV, and/or NSVT analysis may be performed within IMD 16 or within programmer 24. Likewise, the risk stratification indicator may be computed within IMD 16 or within programmer 24.

Processor 100 can take the form one or more microprocessors, DSPs, ASICs, FPGAs, programmable logic circuitry, or the like, and the functions attributed to processor 102 herein may be embodied as hardware, firmware, software or any combination thereof. Memory 102 may store instructions that cause processor 100 to provide the functionality ascribed to programmer 24 herein, and information used by processor 100 to provide the functionality ascribed to programmer 24 herein.

Memory 102 may include any fixed or removable magnetic, optical, or electrical media, such as RAM, ROM, CD-ROM, hard or floppy magnetic disks, EEPROM, or the like. Memory 102 may also include a removable memory portion that may be used to provide memory updates or increases in memory capacities. A removable memory may also allow patient data to be easily transferred to another computing device, or to be removed before programmer 24 is used to program therapy for another patient. Memory 102 may also store information that controls therapy delivery by IMD 40, such as stimulation parameter values.

Programmer 24 may communicate wirelessly with IMD 40, such as using RF communication or proximal inductive interaction. This wireless communication is possible through the use of telemetry module 102, which may be coupled to an internal antenna or an external antenna. An external antenna that is coupled to programmer 24 may be placed over heart 12. Telemetry module 102 may be similar to telemetry module 86 of IMD 16, 40 (FIGS. 3 and 4).

Telemetry module 102 may also be configured to communicate with another computing device via wireless communication techniques, or direct communication through a wired connection. Examples of local wireless communication techniques that may be employed to facilitate communication between programmer 24 and another computing device include RF communication according to the 802.11 or Bluetooth specification sets, infrared communication, e.g., according to the IrDA standard, or other standard or proprietary telemetry protocols. In this manner, other external devices may be capable of communicating with programmer 24 without needing to establish a secure wireless connection.

Power source 108 delivers operating power to the components of programmer 24. Power source 108 may include a battery and a power generation circuit to produce the operating power. In some examples, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within programmer 24. In other examples, traditional batteries (e.g., nickel cadmium or lithium ion batteries) may be used. In addition, programmer 24 may be directly coupled to an alternating current outlet to power programmer 24. Power source 108 may include circuitry to monitor power remaining within a battery. In this manner, user interface 104 may provide a current battery level indicator or low battery level indicator when the battery needs to be replaced or recharged. In some cases, power source 108 may be capable of estimating the remaining time of operation using the current battery.

In some examples, processor 100 may generate a risk stratification indicator based on a BRS measurement and, optionally, at least one of the HRV measurement and NSVT indicator. Again, the BRS measurement, HRV measurement, and/or NSVT indicator may be obtained from IMD 16 or determined by processor 100 based on raw, processed or parametric data obtained from IMD 16. For example, as described in further detail below, processor 80 of IMD 16, 40 may determine the BRS measurement based on an R−R interval difference and a blood pressure difference, such as a right ventricular blood pressure difference. Additionally and optionally, processor 80 may determine at least one of the HRV measurement and the NSVT indicator. Processor 80 then may communicate the BRS measurement, HRV measurement, and/or NSVT indicator to processor 100 via telemetry modules 90 and 102. Processor 100 may generate the risk stratification indicator based on the BRS measurement and the at least one of the HRV measurement and NSVT indicator. In some examples, processor 100 may generate the risk stratification indicator based on the BRS measurement in combination with one or more of the HRV measurement, an ejection fraction measurement, the NSVT indicator, an age of patient 14, gender of patient 14, history of heart failure or cardiac disease of patient 14, or the like.

In other examples, processor 100 of programmer 24 also may determine at least one of the BRS measurement, the HRV measurement, and the NSVT indicator based on raw or parametric signal data communicated from processor 80 to processor 100 via telemetry modules 90 and 102. For example, processor 80 may detect cardiac signals via one or more of electrodes 52, 54, 56, 58, 60, 62, 70, 74, 76, 78, and may detect a blood pressure signal via pressure sensor 34. Processor 80 may transfer the cardiac signals and the blood pressure signals to processor 100 via telemetry modules 90 and 102. Processor 100 may apply one or more techniques described herein to determine at least one of the BRS measurement, the HRV measurement, and the NSVT indicator based on the cardiac signals and/or the blood pressure signals. Processor 100 then may generate the risk stratification indicator based on the BRS measurement and, optionally, at least one of the HRV measurement and the NSVT indicator. In some examples, processor 100 may generate the risk stratification indicator based on the BRS measurement in combination with one or more of the HRV measurement, an ejection fraction measurement, the NSVT indicator, an age of patient 14, gender of patient 14, history of heart failure or cardiac disease of patient 14, or the like.

Processor 100 may also generate an indicator based on the risk stratification indicator. The indicator may include, for example, an implantation indicator, which indicates the patient is a candidate for implantation of an implantable therapy device, such as an implantable cardioverter/defibrillator (ICD), or an implantable drug delivery device. Processor 100 may also automatically initiate, cease, or adjust an existing cardiac therapy delivered by IMD 40 based on the risk stratification indicator. In other examples, processor 100 may generate an alert or alarm to a user, such as patient 14 or a clinician. The alert or alarm may indicate that a condition of patient 14 has changed or is changing.

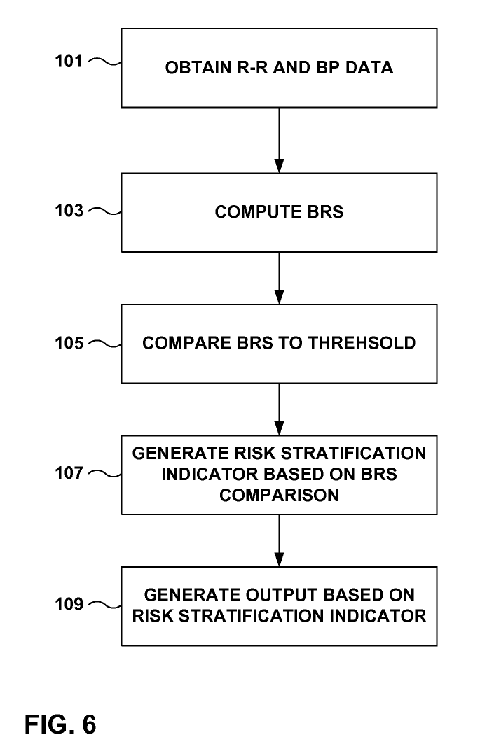

FIG. 6 is a flow diagram illustrating an example technique of generating a risk stratification indicator. Although IMD 16 or IMD 40 may perform the technique illustrated in FIG. 6, IMD 16 will be described for purposes of illustration. As shown in FIG. 6, upon obtaining R−R interval data and blood pressure (BP) data (101), IMD 16 computes a BRS measurement (103). As illustrated in FIGS. 3 and 4, measurement unit 84 of IMD 16 may include pressure sensing module 88 coupled to a pressure sensor and sensing module 86 coupled to one or more electrodes. Measurement unit 84 may receive a blood pressure signal via the pressure sensor and a cardiac signal via one or more electrodes. Using the R−R difference and BP difference over a given period of time, IMD 16 computes the BRS measurement (103).

In other examples, measurement unit 84 may receive a blood pressure signal from a pressure sensor that is not mechanically coupled to IMD 16 and is implanted in a different location of patient 14 from IMD 16. For example, pressure sensor 34 of FIGS. 3 and 4 may be a self-contained implantable device that measures a blood pressure of patient 14 in an artery, vein, or another chamber of heart 12, such as right atrium 26, left atrium 30, or left ventricle 32. In some examples, pressure sensor 34 may include pressure sensing module 88 within the self-contained device, and may communicate the sensed pressure to IMD 16 via wired or wireless communication protocols. In any case, pressure sensor 34 may include any pressure sensing device, including, for example, a capacitive sensor, piezoelectric sensor, mechanical sensor, fiber optic sensor, or the like, and may sense absolute pressure.

Processor 80 may determine the BRS measurement based on the blood pressure signal from pressure sensor 34 or a signal output by pressure sensing module 88 and cardiac signals from sensing module 86. For example, processor 80 may determine a difference in blood pressure (ΔBP) based on the blood pressure signal and a difference in R−R interval (ΔR−R) based on the cardiac signals, as described above and described in further detail below with reference to FIG. 7. Processor 80 then computes the BRS as:

$$B R S = \frac{Δ R - R ( ms )}{Δ B P ( mm Hg )}$$

where ΔR−R is the difference in R−R interval measured in milliseconds (ms) for a given period of time and ΔBP is the difference in blood pressure measured in mm Hg for the same period of time, which may be referred to as a detection interval.

In other examples, processor 80 may compute the BRS measurement by performing linear regression analysis of a plurality of R−R interval and blood pressure pairs (e.g., a blood pressure measurement collected during the same cardiac cycle as the respective R−R interval measurement). For example, processor 80 may determine R−R intervals and blood pressures for a plurality, e.g., five, consecutive cardiac cycles, and may perform linear regression analysis on these five R−R interval and blood pressure pairs. The BRS measurement is the slope of the linear regression line. In this manner, the computation of BRS uses several R−R intervals and corresponding blood pressure points, e.g., over 5 consecutive beats as mentioned above, and calculates a linear regression line through the points. The slope of this line is the BRS value.

Processor 80 also may compute an average BRS measurement from a plurality of BRS measurements, and may store this average in memory 82 as the BRS measurement. For example, processor 80 may average five consecutive BRS measurements into a single average BRS measurement and may store this average BRS measurement in memory 82. In other words, BRS may be computed as the average of n (e.g., n=5) consecutive slope measurements of the ratio of R−R interval differences to blood pressure differences.

Processor 80 then may generate a risk stratification indicator based on the BRS measurement. For example, processor 80 may compare the BRS measurement to a threshold (105) and generate the risk stratification indicator based on the comparison (107). The risk stratification indicator may comprise an indication of the risk of cardiac arrhythmia or cardiac mortality to patient 14, which may be a binary output indicating risk or no risk, or high risk or low risk, or a value that indicates a severity of the risk of cardiac arrhythmia or cardiac mortality. Processor 80 may generate the risk stratification indicator by comparing the BRS measurement to a threshold value (105) or multiple threshold values. For example, clinical studies indicate that a depressed BRS measurement, such as a BRS measurement less than 3 ms/mm Hg, may indicate an increased risk of cardiac mortality among patients with previous myocardial infarction, as described in Maria Teresa La Rovere et al., Baroreflex Sensitivity and Heart Rate Variability in the Identification of Patients at Risk for Life-threatening Arrhythmias: Implications for Clinical Trials, 103 Circulation 2072 (2001).

In other examples, processor 80 may compare the BRS measurement to another threshold value, which may be greater than or less than 3 ms/mm Hg. Processor 80 may generate the risk stratification indicator when the BRS measurement is below the threshold value, which indicates a depressed ability of heart 12 to respond to changes in blood pressure. In some examples, the risk stratification indicator may comprise a binary output, such as risk or no risk, or low risk or high risk, or may comprise one of a plurality of risk levels (e.g., very low risk, medium risk, high risk, or minor cardiac arrhythmia, severe cardiac arrhythmia, cardiac mortality), in which case multiple thresholds for each category are utilized as described below.

The BRS measurement may be compared to a single threshold, such as 3 ms/mm Hg, to produce one of two risk strata, i.e., to classify the patient into one of two cardiac arrhythmia or cardiac mortality risk categories. Alternatively, the BRS may be compared to two or more thresholds, e.g., 2.5 ms/mm Hg and 3.5 ms/mm Hg, to produce three of more risk strata, i.e., to classify the patient into one of three or more cardiac arrhythmia or cardiac mortality risk categories. In this example, a BRS below approximately 2.5 ms/mm Hg may be classified as high risk, a BRS between approximately 2.5 and 3.5 ms/mm Hg may be classified as medium risk, and a BRS above approximately 3.5 ms/mm Hg may be classified as low risk. In this case, processor 80 compares the BRS to a first (high risk) threshold and a second (low risk) threshold. In other implementations, additional thresholds may be used to even more specifically stratify the cardiac arrhythmia or heart failure risk for the patient, e.g., into four, five, six or more cardiac arrhythmia or cardiac mortality risk categories. In each case, the classification can provide a clinician with a convenient and ready guide to the patient's cardiac disease state, facilitating formulation of a course of therapy by the clinician, such as implantation of an IMD such as an ICD. For example, rather than presenting raw BRS values, the cardiac arrhythmia or cardiac mortality risk categories may be expressed textually (e.g., low, medium, high) to permit ready interpretation, in a simple numeric format (e.g., 1, 2, 3 or A, B, C), or in a color-coded format (e.g., green, yellow, red), or in a variety of other ways.

In some examples, processor 80 may generate an output, which may be an indicator, instruction or alert, based on the risk stratification indicator (109). For example, processor 80 may generate an alert to a user, such as patient 14 or a clinician. The alert may indicate that the condition of patient 14 has changed, and may include, for example, an indication of the relative risk to patient 14, or an indication of the measurement that prompted the generation of the risk stratification indicator. For example, the alert may include the value of the BRS measurement, HRV measurement, or NSVT indicator that prompted the generation of the alert to patient 14 or clinician.

In other examples, processor 80 may generate based on the risk stratification indicator an indicator that patient 14 is a candidate for implantation of an IMD that provides therapy, such as stimulation therapy or drug delivery. For example, the implantation indicator may indicate that patient 14 may benefit from an IMD that provides electrical pacing to heart 12 to modify the heart rate in response to a sensed change in blood pressure, in order to moderate the BRS measurement to an acceptable value. As another example, the implantation indicator may indicate that patient 14 may benefit from an IMD that provides stimulation therapy to heart 12 to regulate NSVT or to improve HRV.

In other examples, processor 80 may generate an instruction to initiate or modify a therapy program according to which IMD 40 delivers stimulation to heart 12 based on the risk stratification indicator. For example, the instruction may initiate resetting or suspension of the current therapy program by IMD 40, or may initiate IMD 40 to switch to a different therapy program. Each therapy program may define a plurality of stimulation parameters, including, for example, stimulation pulse width, stimulation pulse amplitude, stimulation frequency, an electrode configuration and/or polarity, or the like.

As an illustration, if the patient is classified into a low risk stratum, IMD 16 or 40 may not generate an alert, but may generate a risk stratification indicator and/or an implantation indicator for retrieval by an external programmer 24. If the patient is classified into a medium risk stratum, IMD 16 or 40 may or may not generate an alert, but again may generate a risk stratification indicator and/or an implantation indicator for retrieval by an external programmer 24. If the patient is classified into a high risk stratum by the risk stratification indicator, IMD 16 or 40 may generate an audible or tactile (e.g., vibratory) alarm and/or transmit an alert, instruction, or implantation indicator message to an external programmer 24. For example, IMD 16 or 40 may activate a piezoelectric buzzer or other device to generate the audible or tactile alarm. In addition, in some cases, if IMD 16 or 40 already provides therapy delivery, the respective IMD may adjust the therapy according to the risk stratification indicator.

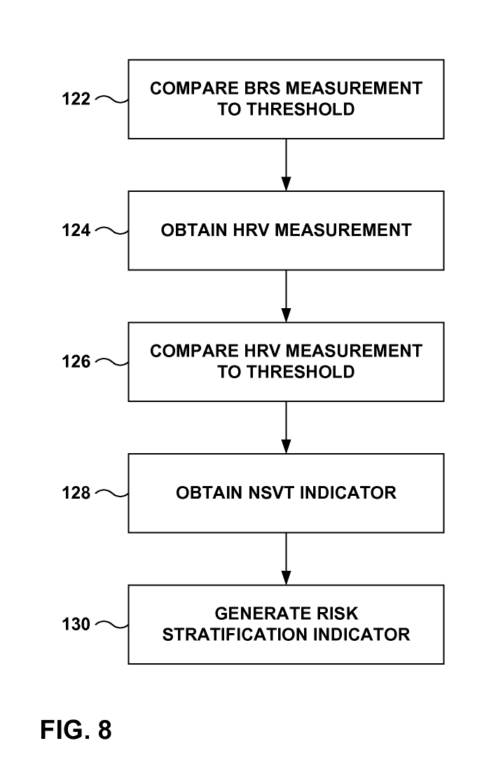

In some examples, processor 80, or alternatively, measurement unit 84, also may obtain at least one of a HRV measurement and a NSVT indicator, as described in further detail below with reference to FIG. 8. Processor 80 then may generate the risk stratification indicator based on the BRS measurement and at least one of the HRV measurement and the NSVT indicator. In some examples, processor 80 may generate the risk stratification indicator based on the BRS measurement in combination with one or more of the HRV measurement, an ejection fraction measurement, the NSVT indicator, an age of patient 14, gender of patient 14, history of heart failure or cardiac disease of patient 14, or the like.

FIG. 7 is a flow diagram illustrating further detail of an example technique according to which IMD 16, and more specifically measurement unit 84 or processor 80, may obtain a BRS measurement. Processor 80 first initiates a detection interval (112) during which processor 80 detects blood pressure signals via pressure sensor 34 and cardiac signals via one or more of electrodes 52, 54, 56, 58, 60, 52, 70, 74, 76, 78, 94. Processor 80 may initiate the detection interval at any time, and in some examples, may initiate the detection interval upon sensing a perturbation to the blood pressure or heart rate of patient 14.

For example, processor 80 may detect a “respiration effect” via a sensor, such as a minute ventilation sensor which measures respiration by monitoring cyclic changes in transthoracic impedance, or electrodes which sense an intracardiac EGM. Hence, in some implementations, IMD 16 or 40 may further include a minute ventilation sensor or be equipped to detect ventilation using intracardiac electrodes. A minute ventilation sensor may include, in part, one or more electrodes deployed on an intracardiac lead, epicardial lead or subcutaneous lead and one or more electrodes deploy on a housing of the IMD, i.e., on the can. Example electrodes include electrodes 52, 54, 56, 58, 60, 52, 70, 74, 76, 78, 94. A sense amplifier within the IMD may monitor signals obtained via such electrodes to determine thoracic impedance and thereby sense ventilation. Cardiac function varies during respiration, a phenomenon referred to as the “respiration effect.” Pressures in the right atrium and thoracic vena cava depend on intrapleural pressure (Pₚₗ). During inspiration, the vagus nerve activity is impeded and heart rate lowers. This causes a fall in Pₚₗ that leads to expansion of the lungs and cardiac chambers (e.g., right atrium and right ventricle), and a reduction in right atrial and ventricular pressures.

As right atrial pressure falls during inspiration, the pressure gradient for venous return to the right ventricle increases. During expiration, the opposite occurs. The degree of heart rate fluctuation is also controlled by regular impulses from the baroreceptors (pressure sensors) in the aorta and carotid arteries as well as cardiopulmonary receptors. Respiration provides a convenient basis for measuring BRS because the perturbation of blood pressure and resulting change in heart rate may be used as inputs for a continuous BRS measurement. The pressure decrease during inspiration typically induces a heart rate increase. The pressure increase during expiration typically induces a heart rate decrease. Hence, BRS may be determined as a measure of the ability of the individual's heart to react to changes in blood pressure during respiration by changing heart rate.

In other examples, processor 80 may detect other perturbations, at which time processor 80 initiates the detection interval. For example, IMD 16 may include or be communicatively coupled to an accelerometer implanted in or carried by patient 14. Processor 80 may receive signals from the accelerometer and determine a posture or activity level from the signals. For example, memory 82 may be programmed with the relative orientation of the accelerometer and patient 14. Thus, by determining the orientation of the accelerometer, processor 80 may determine the posture (or orientation) of patient 14. A change in the posture of patient 14, such as from sitting to standing, or vice versa, may induce a change in heart rate or a change in blood pressure, at which time processor 80 may initiate the detection interval (112).

The accelerometer may also enable processor 80 to determine an activity level of patient 14. For example, when patient 14 is moving, such as walking, jogging, or running, the motion of patient 14 may result in the accelerometer outputting a periodic signal indicative of the rhythmic movement of patient 14. Based on the frequency of the signal output by the accelerometer, and thus the frequency of the motion, processor 80 may determine an activity in which patient 14 is engaged. Activity may also induce a change in heart rate or change in blood pressure, at which time processor 80 may initiate the detection interval (112). Hence, processor 80 may initiate the detection interval in response to a variety of different sensed events. Alternatively, or additionally, in other examples, processor 80 may initiate the detection interval at scheduled times, random times, or pseudo-random times throughout a period of time, such as a day, week or month.

In some examples, in addition to initiating the detection interval, processor 80 may utilize the signals from the accelerometer to enable comparison of the BRS measurements and, optionally, the HRV measurements or NSVT indicators, under similar patient circumstances, such as similar activity levels or postures. In such examples, processor 80 may determine that such circumstances exist when classifying the BRS measurement data for comparison and trending with past BRS measurement data (described below). Different categories of circumstances may include, for example, an activity level of patient 14 and a posture of patient 14. In these examples, processor 80 may separate the BRS measurements and, optionally, the HRV measurements or NSVT indicators, into those taken during periods of activity and periods of inactivity. By doing so, comparisons between BRS measurements, HRV measurements, and NSVT indicators may be made under similar patient circumstances.

Upon initiation of the detection interval (112), processor 80 may detect cardiac signals via one or more of electrodes 52, 54, 56, 58, 60, 52, 70, 74, 76, 78, 94 and sensing module 86. The cardiac signals may include, for example, an intracardiac EGM, which is a graph of electrical activity of heart 12 of patient. The EGM includes a QRS complex, which corresponds to depolarization and contraction of the ventricles. The QRS complex typically includes a Q-wave, and R-wave, and an S-wave. Accordingly, one measure of cardiac cycle length may be the R−R interval, or the time between consecutive R-waves. Processor 80 may determine the R−R interval for each consecutive pair of R-waves during the detection interval.

Processor 80 then may determine an R−R interval difference based on the determined R−R intervals (114). The R−R interval difference may be the difference between the maximum R−R interval in the detection interval and the minimum R−R interval in the detection interval. Processor 80 also may detect blood pressure signals via pressure sensor 34 and pressure sensing module 88. As described above, pressure sensor 34 may be disposed in right ventricle 28 and coupled to lead 18, 42. In other examples, pressure sensor 34 may be disposed in other chambers of heart 12, such as the right atrium 26, left ventricle 32, or left atrium 33.

Pressure sensor 34 may detect one or more blood pressure measurements, such as, for example, RVSP, RVDP, EPAD, or dP/dt from its position in right ventricle 28. In other examples, pressure sensor 34 may detect, for example, a left ventricular systolic pressure (LVSP), a left ventricular diastolic pressure (LVDP), a left ventricular pulse pressure (LVPP), a left atrial pressure (LAP), or a right atrial pressure (RAP). Processor 80 may use any of these pressures in determining the BRS measurement.

In many examples, a BRS measurement is determined using left ventricular pressures. However, implantation of medical devices, such as pressure sensor 34, in the left ventricular may by disfavored in many cases, because left ventricular implantation may increase risk of blood clot formation and damage to heart 12 or other vascular structures. Thus, in many examples, implantation of pressure sensor 34 in right ventricle 28 may be favored. RV pressures also may be used to determine the BRS measurement.

In some examples, such as those described with respect to FIGS. 1-4, pressure sensor 34 may be disposed in right ventricle 28. In these examples, processor 80 may utilize a RV pressure, such as RVSP, to determine the BRS measurement. More specifically, processor 80 may detect RV pressure for the length of the detection interval. Processor 80 may then determine the RVSP for each heart cycle (e.g., R−R interval) by determining the maximum pressure for each beat. Processor 80 then may determine a difference between the maximum RVSP and the minimum RVSP during the detection interval (116), and use this difference in determining the BRS measurement, as described below.

Once processor 80 has determined the R−R interval difference (114) and determined the blood pressure difference (116), processor 80 determines the BRS measurement (1 18). In examples in which pressure sensor 34 is located in the right ventricle, processor 80 may determine the BRS measurement, as described above, by dividing the difference in R−R intervals for the detection interval by the difference in RVSP for the detection interval:

$$B R S = \frac{Δ R - R ( ms )}{Δ RSVP ( mm Hg )}$$

where ΔR−R is the difference in the maximum R−R interval and the minimum R−R interval for the detection interval and ΔRVSP is the difference in the maximum RV systolic pressure and the minimum RV systolic pressure for the same detection interval. Hence, in this example, the blood pressure difference is a right ventricular blood pressure difference. Processor 80 may proceed to use the BRS measurement to generate the risk stratification indicator (107), as described above with respect to FIG. 6. The value of the risk stratification indicator may be used to automatically generate output (109) such as an implantation indicator. Again, in some examples, the risk stratification indicator may simply comprise a binary output, such as risk or no risk, or high risk or low risk, thereby categorizing the patient into one or two or more cardiac arrhythmia or cardiac mortality risk categories. Alternatively, the risk stratification indicator may have any of multiple values and indicate one of a plurality of different risk levels (e.g., low risk, medium risk, high risk or very low risk, low risk, medium risk, high risk or very high risk), permitting categorization of the patient into three of more cardiac arrhythmia or cardiac mortality risk categories. In turn, IMD 16 or programmer 24 may automatically generate a binary implant indicator such as implant or no implant, or a range of implant indicators such as implant critically needed, patient would benefit from implant, implant not needed but may be beneficial, implant not needed but optional, or no implant benefit likely.

In other examples, processor 80 may compute the BRS measurement by performing linear regression analysis of a plurality of R−R interval and blood pressure pairs (e.g., a blood pressure measurement collected during the same cardiac cycle as the respective R−R interval measurement). For example, processor 80 may determine R−R intervals and blood pressures for a plurality, e.g., five, consecutive cardiac cycles, and may perform linear regression analysis on these five R−R interval and blood pressure pairs. The BRS measurement is the slope of the linear regression line. In this manner, the computation of BRS use several R−R intervals and corresponding blood pressure points, e.g., over 5 consecutive beats as mentioned above, and calculates a linear regression line through the points. The slope of this line is the BRS value.

Processor 80 also may compute an average BRS measurement from a plurality of BRS measurements, and may store this average in memory 82 as the BRS measurement. For example, processor 80 may average five consecutive BRS measurements into a single average BRS measurement and may store this average BRS measurement in memory 82. In other words, BRS may be computed as the average of n (e.g., n=5) consecutive slope measurements of the ratio of R−R interval differences to blood pressure differences.

In the above examples, the difference in the maximum R−R interval and the minimum R−R interval and the difference in the maximum RV systolic pressure and the minimum RV systolic pressure are determined for substantially the same period of time, i.e., substantially the same detection interval. However, BRS can be determined over many different time periods. For example, in one example, when respiration is used as the perturbation that initiates the detection interval, the BRS measurement may be calculated for each respiration cycle. That is, the maximum and minimum R−R intervals and RVSP are determined for each respiration cycle.

In other examples, the BRS measurement may be calculated over only the inspiration period or only the expiration period. The BRS measurement may also be estimated on a beat-to-beat basis at every cardiac cycle using the equation provided above. In such an example, ΔR−R and ΔRVSP are values calculated from the two most recent samples of the R−R interval and RVSP, and it is not necessary to determine respiration cycles prior to calculating the BRS measurement. As noted above, pressure changes other than RVSP may be used. Such pressure changes may be substituted for ΔRVSP in the equation above. In certain examples, ΔR−R and ΔRVSP in the BRS calculation can be measured over different time periods, such as different respiration cycles, different detection intervals, or different periods within a detection interval, as indicated by the following equation:

$$B R S = \frac{{( Δ R - R )}_{t - n , n = 0 , 1 , 2 , 3 , …}}{{( Δ RSVP )}_{t - m , m = 0 , 1 , 2 , 3 , …}}$$

In these examples, (ΔR−R)ₜ₋ₙ represents the difference in the maximum R−R interval and the minimum R−R interval for the time period (t−n), where t is the current time period and n (which may be equal to 0, 1, 2, 3, . . . ) is the number of time periods ago in which the ΔR−R value should be calculated. Similarly, (ΔRVSP)ₜ₋ₘ represents the difference in the maximum RVSP for time period (t−m), where t is again the current respiration cycle and m (which may be equal to 0, 1, 2, 3, . . . ) is the number of time periods ago in which the ΔRVSP should be calculated. Basing the BRS measurement on different respiration cycles (i.e., n not equal to m) provides a BRS measurement that accounts for delays between a change in one variable and an effect on the other variable.

For instance, as noted above, respiration causes pressure changes such as a change in RVSP. It may take several respiration cycles for the pressure change to physiologically induce a heart rate change, such as a change in R−R interval. Using the equation above with, for instance n=0 and m=3, the calculation of the BRS measurement can account for a delay of 3 respiration cycles between the change in R−R interval induced in the (t-0) current respiration cycle the change in RVSP from the (t-3) respiration cycle 3 cycles ago. The values of m and n may be predetermined, preprogrammed in memory 82, or set to change dynamically based on data from sensors communicatively coupled to IMD 16 (i.e., processor 80).

Other perturbations, such as activity or posture, may similarly cause a change in blood pressure (e.g., RVSP) that takes time to affect heart rate. Thus, while respiration may not be measured in these examples, processor 80 may determine the difference in blood pressure and the time period used for different time periods. For example, processor 80 may determine the difference in blood pressure based on cardiac signals that are a certain number of heart cycles prior to the cardiac signals used by processor 80 to determine the difference in R−R interval. The number of heart cycles may be predetermined, preprogrammed in memory 82, or determined dynamically by processor 80.

FIG. 8 is a flow diagram illustrating an example technique according to which processor 80 may generate the risk stratification indicator. Processor 80 may first compare the BRS measurement to one or more thresholds (122). As described above, a depressed BRS measurement may indicate a reduced ability of heart 12 to respond to changes in blood pressure. This reduced ability of heart 12 to respond to changes in blood pressure may indicate a heart condition and an increased risk of cardiac arrhythmia or cardiac mortality.

In some examples, the BRS measurement threshold or thresholds may be predetermined, preprogrammed into memory 82, or set to change dynamically based on data from sensors communicatively coupled to IMD 16. For example, the threshold value(s) may be determined based on prior clinical trials, and may be programmed into memory 82. The threshold value(s) may be, for example, 3 ms/mm Hg, a value greater than 3 ms/mm Hg, or a value less than 3 ms/mm Hg. Also, in some implementations, multiple thresholds may be used to provide three of more BRS risk strata. In the binary example of high and low risk strata as a set of two cardiac arrhythmia or cardiac mortality risk categories, when processor 80 compares the BRS measurement to the threshold value and the BRS measurement falls below the threshold value, processor 80 may interpret this as indicating an increased risk of cardiac arrhythmia or cardiac mortality, and when the BRS measurement falls above the threshold value, processor may interpret this as indicating no increased risk of cardiac arrhythmia or cardiac mortality.

Processor 80 also may optionally obtain an HRV measurement utilizing the cardiac signals, such as an intracardiac EGM (124). Processor 80 may obtain the HRV measurement by calculating a standard deviation of a plurality of consecutive R−R intervals, as described above. For example, processor 80 may determine a standard deviation of R−R intervals of about 15 consecutive R-waves. In other examples, processor 80 may determine the standard deviation of R−R intervals of a greater or fewer number of consecutive R-waves. For instance, processor 80 may determine the standard deviation of R−R intervals of consecutive R-waves for substantially the entire detection interval.

Once processor 80 has obtained the HRV measurement, processor 80 may compare the HRV measurement to a threshold value to determine whether the HRV measurement indicates an increased risk of cardiac arrhythmia or cardiac mortality (126). For example, an HRV measurement of less than about 70 ms may indicate an increased of cardiac arrhythmia or cardiac mortality among patients with previous myocardial infarction, while a HRV measurement of greater than 70 ms may indicate absence of increased risk. In other examples, the threshold value for the HRV measurement may be a value greater than about 70 ms or a value less than about 70 ms. The threshold value for the HRV measurement may be predetermined, preprogrammed into memory 82, or set to change dynamically based on data from sensors communicatively coupled to IMD 16. For example, the threshold value may be determined based on prior clinical trials, and may be programmed into memory 82.

Processor 80 may also optionally determine a NSVT indicator based on the cardiac signal (128). For example, processor 80 may determine an R−R interval for a plurality of consecutive pairs of R-waves. In some examples, processor 80 may determine the R−R interval for each consecutive pair of R-waves for the duration of the detection interval. Processor 80 may then compare R−R interval to a threshold time, such as, for example, 600 ms or less. When processor 80 determines that three consecutive R−R intervals are less than 600 ms, processor 80 may generate the NSVT indicator. The presence of NSVT may indicate an increased of cardiac arrhythmia or cardiac mortality among patients with previous myocardial infarction.

In some examples, processor 80 may obtain the BRS measurement, the HRV measurement, and the NSVT indicator for the same detection interval. In other examples, however, processor 80 may obtain at least one of the BRS measurement, the HRV measurement, and the NSVT indicator for a different detection interval than at least another of the BRS measurement, the HRV measurement, and the NSVT indicator. For example, processor 80 may obtain a NSVT indicator from one detection interval, or a timer period outside of a detection interval, and store the NSVT indicator in memory 82. Processor 80 may use the NSVT indicator, which indicates the presence of NSVT, for subsequent determinations of the risk stratification indicator along with a BRS measurement and, optionally, a HRV measurement obtained for a different previous detection interval or a current detection interval. Similarly, processor 80 may obtain a HRV measurement from one detection interval, or a timer period outside of a detection interval, and store the HRV measurement in memory 82. Processor 80 may use the HRV measurement, which indicates the presence or absence of HRV, for subsequent determinations of the risk stratification indicator along with a BRS measurement and, optionally, a HRV measurement obtained for a different previous detection interval or a current detection interval. In some examples, each of the BRS measurement, the HRV measurement, and the NSVT indicator may be from different detection intervals.

Processor 80 then generates the risk stratification indicator (130). In some examples, processor 80 may generate the risk stratification indicator based solely on the BRS measurement. In other examples, processor 80 may determine the risk stratification indicator based on two or more of the BRS measurement, the HRV measurement, and the NSVT measurement. Determination of the risk stratification indicator based on two or more of the BRS measurement, the HRV measurement, and the NSVT measurement may improve, for example, the specificity of the positive and negative predictive values of the risk stratification indicator or may allow processor 80 to more readily generate a risk stratification indicator with more than two levels (e.g., risk or no risk).

In some examples, processor 80 may generate the risk stratification indicator based on the BRS measurement in combination with other parameters, such as, for example, an ejection fraction measurement, an age of patient 14, gender of patient 14, or a history of heart failure or cardiac disease of patient 14. Memory 82 may store the parameters, which may have been transmitted to IMD 16 by a clinician via programmer 24. Processor 80 then may generate the risk stratification indicator based on the BRS measurement in combination with one or more of the HRV measurement, an ejection fraction measurement, the NSVT indicator, an age of patient 14, gender of patient 14, history of heart failure or cardiac disease of patient 14, or the like.

Processor 80 may generate a risk stratification indicator that comprises an indication of whether any of the BRS measurement, the HRV measurement, and the NSVT measurement indicates an increased risk of cardiac arrhythmia or cardiac mortality to patient 14. For example, processor 80 may simply generate an indicator that indicates whether the BRS measurement falls above or below the threshold BRS value. Processor 80 may generate a similar indicator for the HRV measurement, if determined, and the NSVT measurement, if determined. The risk stratification indicator, then, may comprise a combination of each of these individual indicators of the presence or absence of a BRS, HRV, or NSVT measurement that indicates an increased risk to patient 14. In some cases, the risk stratification indicator may have a value that indicates one of several different risk levels.

In some examples, processor 80 may generate a risk stratification indicator that stratifies the risk of cardiac arrhythmia or cardiac mortality into a plurality of levels. As mentioned above, processor 80 may compare a single parameter such as BRS to multiple thresholds. Alternatively, presence or absence of a depressed BRS measurement, a depressed HRV measurement, and NSVT may each contribute to a count or summation, and processor 80 may assign a risk stratification indicator based on the count or summation. For example, presence of depressed BRS and HRV measurements and NSVT, i.e., all three, may cause processor 80 to generate a risk stratification indicator that classifies the patient into a cardiac arrhythmia or cardiac mortality risk category that corresponds a very high risk of cardiac arrhythmia or cardiac mortality.

Continuing the example, presence of two of the three indicators may cause processor 80 to generate a risk stratification indicator that indicates a high risk of cardiac arrhythmia or cardiac mortality, presence of a single indicator may cause processor 80 to generate a risk stratification indicator that indicates a medium risk of cardiac arrhythmia or cardiac mortality, and absence of all indicators may cause processor 80 to generate a risk stratification indicator that indicates a low risk of cardiac arrhythmia or cardiac mortality. In other examples, presence of a certain number of indicators and may result in processor 80 generating a different risk stratification indicator. For example, presence of two or three indicators may indicate a high risk, presence of a single indicator may indicate a medium risk and absence of all indicators may indicate a very low risk.

Processor 80 may also generate different risk stratification indicators for the presence of depressed BRS and HRV measurements and the presence of a depressed BRS measurement and NSVT, as each may represent a different risk to patient 14. For example, the presence of a depressed BRS and HRV measurements may indicate a lower risk to patient 14 than the presence of a depressed BRS measurement and NSVT. Accordingly, processor 80 may generate a risk stratification indicator that indicates a lower risk of cardiac arrhythmia or cardiac mortality when depressed BRS and HRV measurements are detected than when a depressed BRS measurement and NSVT are present.

In other examples, processor 80 may utilize at least two of the BRS, HRV, and NSVT measurements in a weighted summation technique to generate a risk stratification indicator. For example, one of the BRS, HRV, and NSVT measurements may contribute more to the risk to patient 14 than at least one other of the BRS, HRV, and NSVT measurements. This measurement may be weighted more heavily in the summation in order to reflect the increased risk of cardiac arrhythmia or cardiac mortality. Conversely, one of the BRS, HRV, and NSVT measurements may contribute less to the risk of cardiac arrhythmia or cardiac mortality to patient 14 than at least one other of the BRS, HRV, and NSVT measurements, and this measurement may be weighted less heavily in the summation in order to reflect this fact.

Hence, each of the indicators may carry equal weight in the summation. In other cases, some of the indicators may be weighted more heavily than others in the summation. As an illustration, a risk stratification indicator could be computed based on a weighted sum of the BRS, HRV and NSVT indicators, e.g., Risk Score=m₁BRS+m₂HRV+m₃NSVT. In some examples, if BRS is the considered the most conclusive or most important indicator of cardiac arrhythmia or cardiac mortality risk, m₁ may be greater than m₂ and m₃. Then, the total Risk Score may be compared to one or more thresholds to classify the risk and generate the risk stratification indicator. For example, processor 80 may compare the value resulting from the weighted summation to a scale, table, threshold or multiple thresholds to determine the risk level indicated by the value, and determine the risk stratification indicator based on the risk level indicated by the scale or table. In some implementations, instead of a strict classification of the risk into one or two or more risk strata, the risk stratification indicator may take the form of a numerical score, which may be meaningful to a clinician or may be used to automatically generate an implantation input.

In some examples, processor 80 may cause the BRS measurement, HRV measurement, NSVT indicator, and/or risk stratification indicator to be stored in memory 82 to create a measurement history. Processor 80 may utilize the measurement history to monitor a trend in at least one of the measurements or indicators. For example, processor 80 may cause the BRS measurement to be stored in memory 82 and may use the BRS measurement history to monitor a trend of the BRS measurements over time. For instance, processor 80 may monitor the BRS measurement trend to determine whether the measurements remain above a lower threshold or within an envelope of predetermined upper and lower thresholds, such as, for example, a lower threshold of 3 ms/mm Hg and, optionally, an upper threshold of 4 ms/mm Hg. The upper and lower thresholds described herein are merely exemplary, and it will be understood that the thresholds may be determined for each patient 14 based on, for example, initial BRS measurements of patient 14, dynamically changing thresholds calculated by processor 80 based on a previously obtained BRS measurements, or clinical tests performed on a group of patients.

BRS measurements may be stored in memory 82 and trended over a period of time, or indefinitely. Various techniques can be employed to compose the trend data. For example, trend data points may be calculated median or mean values of any give time duration, or the data points could be smoothed via a low-pass filter smoothing function. Processor 80 may analyze the trend to determine whether it decreases below the lower threshold, which may indicate the condition of patient 14 may have deteriorated to a clinically significant level that may necessitate further investigation or remedial action.

In some examples, processor 80 may generate a risk stratification indicator when the trend crosses the lower threshold. The risk stratification indicator may trigger, for example, delivery of an alert to patient 14 or a clinician. Processor 80 may communicate the alert to programmer 24 or another computing device via telemetry module 90. The risk stratification indicator may also trigger, for example, delivery of a therapy, such as delivery of electrical stimulation therapy to heart 12 or delivery of a drug to patient 14 or modification of a therapy that is already being delivered to patient 14. As examples, IMD 16 may deliver or adjust right atrial pacing, right ventricular pacing, cardioversion shocks, defibrillation shocks, CRT, CPT, or PESP therapy, or drug delivery in response to the generation of the risk stratification indicator. As a particular example, IMD 16 may modify a drug dosage based on the risk stratification indicator. Such therapy adjustments or deliveries may be based on the generation of the risk stratification indicator or based on a level or score of the risk stratification indicator.

By utilizing trending of the measurements and/or indicators, one measurement or indicator below the lower threshold or above the upper threshold may not cause processor 80 to generate the risk stratification indicator. This may reduce the number of false alerts communicated to patient 14 or a clinician, or may reduce the number of unnecessary therapy deliveries or therapy adjustments, while still allowing alerts or therapy adjustments to be generated upon determination of an increased risk of cardiac arrhythmia or cardiac mortality to patient 14.

While the above discussion of trending was primarily directed to BRS measurements, processor 80 may also store and monitor or trend HRV measurements, NSVT indicators, and/or risk stratification indicators. For example, processor 80 may trend HRV measurements and determine whether the trendline of the HRV measurements crosses a lower or upper threshold value, similar to the technique described with respect to BRS measurements. In other examples, processor 80 may cause NSVT indicators to be stored in memory 82, or may maintain a count of NSVT indicators. Upon reaching a threshold number of NSVT indicators, processor 80 may generate a risk stratification indicator that causes processor 80 to generate an alert to patient 14 or a clinician, a modification of a therapy being delivered to patient 14, or a delivery of therapy to patient 14. In some cases, processor 80 may be configured to automatically generate an implantation indicator based on the risk stratification indicator.

In still other examples, processor 80 may cause two or more of BRS measurements, HRV measurements, and NSVT indicators to be stored in memory 82. Processor 80 may maintain a count of the BRS measurements, HRV measurements, and NSVT indicators or may trend BRS measurements, HRV measurements, and NSVT indicators. For instance, processor 80 may trend the number of measurements/indicators present, and may generate a risk stratification indicator when a number of measurements/indicators increases. For example, initially, depressed BRS may be present, but not depressed HRV or NSVT. Subsequently, processor 80 may determine that depressed HRV is present, and may generate a risk stratification indicator because of the increase in the number of measurements/indicators detected. Once again, the risk stratification indicator may trigger processor 80 to generate an alarm or alert to patient 14 or a clinician, a modification of a therapy being delivered to patient 14, or a delivery of therapy to patient 14.

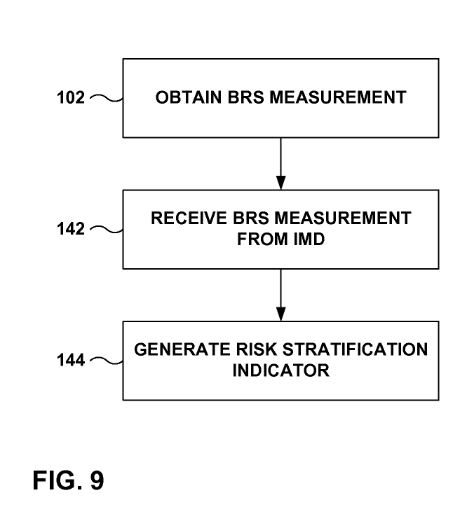

While the previous discussion has focused on an IMD 16 which both obtains a BRS measurement and generates a risk stratification indicator, in other examples, programmer 24 (e.g., processor 100) may contribute to at least one of obtaining the BRS measurement and generating the risk stratification indicator. For example, FIG. 9 is a flow diagram of a technique in which processor 100 generates the risk stratification indicator.

Initially, processor 80, or alternatively measurement unit 84, obtains a BRS measurement (102) as described in further detail above with respect to FIGS. 6 and 7. In some examples, processor 80 also may determine a HRV measurement and NSVT indicator. Processor 80 then communicates the BRS measurement and, optionally, the HRV measurement and NSVT indicator to processor 100 via telemetry modules 90 and 106. Processor 100 receives the BRS measurement and, optionally, HRV measurement and NSVT indicator, from processor 80 of IMD 16 (142) and generates the risk stratification indicator (144), as shown in FIG. 9. Processor 80 may generate the risk stratification indicator based on, for example, a comparison of the BRS measurement to a threshold value or multiple threshold values. Processor 100 may generate the risk stratification indicator based on the BRS measurement and, optionally, at least one of the HRV measurement and NSVT indicator as described in further detail above. For example, processor 100 may count the number of measurements and indicators present, may calculate a weighted summation of the measurements and indicators, or trend the BRS measurement and, optionally, the HRV measurement and NSVT indicator. In some examples, the risk stratification indicator may comprise a binary output, such as risk or no risk, or may comprise one of a plurality of cardiac arrhythmia or cardiac mortality risk categories (e.g., very low risk, medium risk, high risk) with any of a variety of different granularity levels.

Again, processor 100 may generate, for example, an alert to a user, such as patient 14 or a clinician, based on the risk stratification indicator. The alert may indicate that the condition of patient 14 has changed, and may include, for example, an indication of the relative risk to patient 14, or an indication of the measurement that prompted the generation of the risk stratification indicator. For example, the alert may include the value of the BRS measurement, HRV measurement, or NSVT indicator that prompted the generation of the alert to patient 14 or clinician. If the risk stratification indicator is unchanged or indicates a low risk, there may not need to generate an alarm or alert. However, the risk stratification indicator may be transmitted to programmer 24 or stored in IMD 16 for later retrieval by programmer 24.

In other examples, processor 100 may generate based on the risk stratification indicator an indicator that patient 14 is a candidate for implantation of an IMD that provides therapy, such as stimulation therapy or drug delivery. A patient may be considered a candidate for implantation of the device if the risk stratification indicator indicates that the patient is critically in need of the device or would benefit from the device in order to reduce cardiac arrhythmia or cardiac mortality risk. For example, the implantation indicator may indicate that patient 14 may benefit from an IMD that provides electrical pacing or cardioversion/defibrillation therapy to heart 12 to modify the heart rate in response to a sensed change in blood pressure, in order to moderate the BRS measurement to an acceptable value. As another example, the implantation indicator may indicate that patient 14 may benefit from an IMD that provides stimulation therapy to heart 12 to regulate a NSVT or to improve HRV. In general, processor 100 may automatically generate a binary implant indicator such as implant or no implant, or a range of implant indicators such as implant critically needed, patient would benefit from implant, implant not needed but may be beneficial, implant not needed but optional, or no implant benefit likely. Different values of the risk stratification indicator may be mapped to different implant indicators. Hence, processor 100 may generate different implant indications for different, corresponding values of the risk stratification indicator.

In other examples, processor 100 may generate an instruction to initiate or modify a therapy program according to which IMD 40 delivers stimulation to heart 12 based on the risk stratification indicator. For example, the instruction may initiate resetting or suspension of the current therapy program by IMD 40, or may initiate IMD 40 to switch to a different therapy program. Each therapy program may define a plurality of stimulation parameters, including, for example, stimulation pulse width, stimulation pulse amplitude, stimulation frequency, an electrode configuration and/or polarity, or the like. Processor 100 may communicate the instruction to processor 80 via telemetry modules 90 and 106, and processor 80 may control stimulation generator 98 to cause the appropriate response indicated by the instruction. When the risk stratification indicator results in generation of an indication that the patient is a candidate for an IMD or drug therapy, or an indication that current therapy already provided to the patient should be adjusted, programmer 24 may present a message proposing such action to the clinician. In response, the clinician may voluntarily take the recommended action. In the case of proposed adjustments to electrical stimulation therapy, programmer 24 may be configured to present particular programs or parameter adjustments for approval by the clinician. If the clinician approves the programs or adjustments, programmer 24 may communicate them to IMD 16 or 40 and direct the IMD to implement them in therapy delivered to the patient.

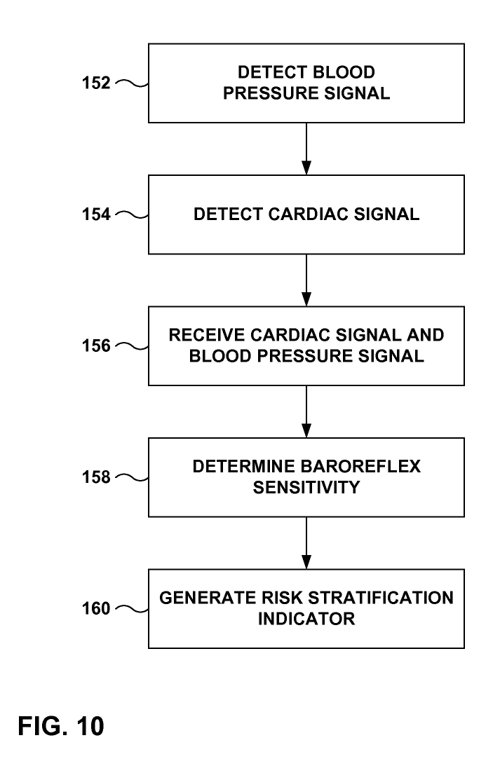

FIG. 10 is a flow diagram of an alternate technique according to which processor 100 of programmer 24 may generate a risk stratification indicator. Initially, processor 80, or alternatively, measurement unit 84, detects a blood pressure signal (152) and a cardiac signal (154). As described above, processor 80 may detect the blood pressure signal via pressure sensor 34 and pressure sensing module 88 and the cardiac signal via one or more of electrodes 52, 54, 56, 58, 60, 62, 70, 74, 78, 94, and cardiac sensing module 86.

In some examples, processor 80 may apply one or more data processing techniques to the blood pressure signal and/or the cardiac signal, such as, for example, analog to digital conversion, high or low pass filtering, formatting, or the like. Processor 80 then transmits the blood pressure signal and cardiac signal to processor 100 of programmer 24 via telemetry modules 90 and 106. Processor 100 receives the blood pressure signal and cardiac signal (156).

Processor 100 then may determine the BRS measurement based on the blood pressure signal and cardiac signal (158), as described in further detail above. In some examples, processor 100 may also determine at least one of a HRV measurement and a NSVT indicator based on the cardiac signal. Processor 100 then may generate a risk stratification indicator based on the BRS measurement and, optionally, at least one of the HRV measurement and the NSVT indicator (160). As described above, processor 100 may generate based on the risk stratification indicator, for example, an alert to patient 14 or a clinician, an initiation or modification of therapy delivered to patient 14, or an indication that patient 14 is a candidate for an IMD that delivers therapy. The risk stratification indicator also may comprise a binary output (e.g., risk or no risk), or one of a plurality of cardiac arrhythmia or cardiac mortality risk categories (e.g., very low risk, medium risk, high risk).

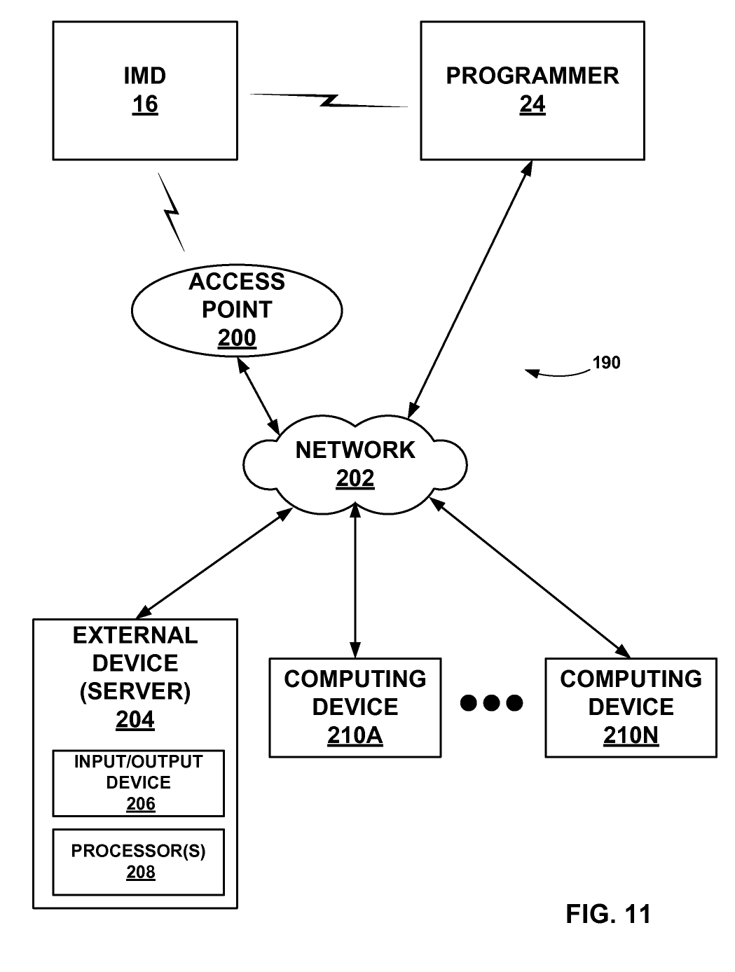

FIG. 11 is a block diagram illustrating an example system 190 that includes an external device, such as a server, and one or more computing devices that are coupled to the IMD and programmer shown in FIG. 1 via a network. In some implementations, physiological signal data may be transmitted from IMD 16 or 40 to programmer 24 or another device and, in turn, to a server and/or client computers coupled to programmer 24 or the other device via a network. In this case, a remote server may compute BRS, HRV and/or NSVT indicators and/or compute a risk stratification indicator based on information received from IMD 16 or 40 and/or programmer 24. Alternatively, BRS, HRV, and/or NSVT indicators and/or risk stratification indicators generated by IMD 16 or 40 or programmer 24 may be transmitted to such a remote server or client computer for processing, archival and/or viewing by a clinician or other caregiver.

In the example of FIG. 11, example system 190 includes an external device, such as a server 204, and one or more client computing devices 210A-210N, that are coupled to the IMD 16 and programmer 24 shown in FIG. 1 via a network 202. In this example, IMD 16 may use its telemetry module 88 to communicate with programmer 24 via a first wireless connection, and to communicate with an access point 200 via a second wireless connection. In the example of FIG. 11, access point 200, programmer 24, server 204, and computing devices 210A-210N are interconnected, and able to communicate with each other, through network 202.

In some cases, one or more of access point 200, programmer 24, server 204, and computing devices 210A-210N may be coupled to network 202 through one or more wireless connections. IMD 16, programmer 24, server 204, and computing devices 210A-210N may each comprise one or more processors, such as one or more microprocessors, DSPs, ASICs, FPGAs, programmable logic circuitry, or the like, that may perform various functions and operations, such as those described herein. For example, as illustrated in FIG. 11, server 204 may comprise one or more processors 208 and an input/output device 206, which need not be co-located.

Server 204 may, for example, implement any of the methods described herein for generation of a risk stratification indicator, including generation of the risk stratification indicator itself and any intermediate operations, such as generating BRS, HRV and/or NSVT indicators from raw, processed or parametric pressure signals and cardiac signals, marker channel data, or other information. Server 204 also may provide a database or other memory for storing such information.

Access point 200 may comprise a device that connects to network 202 via any of a variety of connections, such as telephone dial-up, digital subscriber line (DSL), or cable modem connections. In other embodiments, access point 200 may be coupled to network 202 through different forms of connections, including wired or wireless connections. In some embodiments, access point 200 may be co-located with patient 14 and may comprise one or more programming units and/or computing devices (e.g., one or more monitoring units) that may perform various functions and operations described herein. For example, access point 200 may include a home-monitoring unit that is co-located with patient 14 and that may monitor the activity of IMD 16. In some embodiments, server 204 or one or more of the computing devices 210A-210N may perform any of the various functions or operations described herein.

Network 202 may comprise a local area network, wide area network, or global network, such as the Internet. In some cases, programmer 24 or server 204 may assemble BRS, HRV, NSVT or risk stratification indicators or data in web pages or other documents for viewing by trained professionals, such as clinicians, via viewing terminals associated with computing devices 210A-210N. System 190 may be implemented, in some aspects, with general network technology and functionality similar to that provided by the Medtronic CareLink® Network developed by Medtronic, Inc., of Minneapolis, Minn.

The techniques described in this disclosure, including those attributed to ICD 16 or various constituent components, may be implemented, at least in part, in hardware, software, firmware or any combination thereof. For example, various aspects of the techniques may be implemented within one or more processors, including one or more microprocessors, digital signal processors (DSPs), application specific integrated circuits (ASICs), field programmable gate arrays (FPGAs), or any other equivalent integrated or discrete logic circuitry, as well as any combinations of such components, embodied in programmers, such as physician or patient programmers, stimulators, or other devices. The term “processor” or “processing circuitry” may generally refer to any of the foregoing circuitry, alone or in combination with other circuitry, or any other equivalent circuitry.

Such hardware, software, or firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

When implemented in software, the functionality ascribed to the systems, devices and techniques described in this disclosure may be embodied as instructions on a computer-readable medium such as random access memory (RAM), read-only memory (ROM), non-volatile random access memory (NVRAM), electrically erasable programmable read-only memory (EEPROM), FLASH memory, magnetic data storage media, optical data storage media, or the like. The instructions may be executed to support one or more aspects of the functionality described in this disclosure.

Various examples have been described. These and other examples are within the scope of the following claims.

1. A method comprising:
obtaining a baroreflex sensitivity (BRS) measurement for a patient via an implantable medical device (IMD); and
generating a risk stratification indicator based on the BRS measurement, wherein the risk stratification indicator classifies the patient into one of a plurality of cardiac arrhythmia or cardiac mortality risk categories.

2. The method of claim 1, further comprising generating based on the risk stratification indicator an implantation indicator that indicates the patient is a candidate for implantation of an implantable therapy device.

3. The method of claim 2, wherein generating based on the risk stratification indicator the implantation indicator comprises generating an implantation indicator that indicates the patient is a candidate for an implantable cardioverter-defibrillator.

4. The method of claim 1, wherein generating the risk stratification indicator comprises generating the risk stratification indicator via at least one of the IMD and an external computing device.

5. The method of claim 1, wherein generating the risk stratification indicator comprises generating an indicator of at least one of low risk, medium risk, and high risk.

6. The method of claim 1, further comprising obtaining a heart rate variability (HRV) measurement via the IMD, and wherein generating the risk stratification indicator based on the BRS measurement comprises generating the risk stratification indicator based on the BRS measurement and the HRV measurement.

7. The method of claim 6, further comprising obtaining a non-sustained ventricular tachycardia (NSVT) measurement via the IMD, and wherein generating the risk stratification indicator based on the BRS measurement comprises generating the risk stratification indicator based on the BRS measurement, the HRV measurement, and the NSVT measurement.

8. The method of claim 1, further comprising obtaining a non-sustained ventricular tachycardia (NSVT) measurement via the IMD, and wherein generating the risk stratification indicator based on the BRS measurement comprises generating the risk stratification indicator based on the BRS measurement and the NSVT measurement.

9. The method of claim 1, wherein obtaining the BRS measurement via the IMD comprises:
determining an R−R interval difference indicating a time between successive ventricular depolarizations;
determining a blood pressure difference; and
determining the BRS measurement based on the R−R interval difference and the blood pressure difference.

10. The method of claim 9, wherein the blood pressure difference is a right ventricular blood pressure difference.

11. The method of claim 9, further comprising communicating the R−R interval difference and the blood pressure difference to a computing device, wherein the computing device determines the BRS measurement based on the R−R interval difference and the blood pressure difference.

12. An implantable medical device (IMD) comprising:
a measurement unit configured to obtain a baroreflex sensitivity (BRS) measurement for a patient; and
a processor that generates a risk stratification indicator based on the BRS measurement, wherein the risk stratification indicator classifies the patient into one of a plurality of cardiac arrhythmia or cardiac mortality risk categories.

13. The IMD of claim 12, wherein the processor generates based on the risk stratification indicator an implantation indicator that indicates the patient is a candidate for an implantable therapy device.

14. The IMD of claim 13, wherein the processor generates based on the risk stratification indicator an implantation indicator that indicates the patient is a candidate for an implantable cardioverter-defibrillator.

15. The IMD of claim 12, wherein the risk stratification indicator comprises an indicator of at least one of low risk, medium risk, and high risk of cardiac arrhythmia or cardiac mortality.

16. The IMD of claim 12, wherein the measurement unit comprises one or more electrodes that detect a cardiac signal and a pressure sensor and detects a blood pressure signal, and wherein the processor is configured to receive the cardiac signal and the blood pressure signal and determine the BRS measurement.

17. The IMD of claim 16, wherein the processor is further configured to determine a heart rate variability (HRV) measurement based on the cardiac signal and generate the risk stratification indicator based on the BRS measurement and the HRV measurement.

18. The IMD of claim 16, wherein the processor is further configured to determine a non-sustained ventricular tachycardia (NSVT) measurement based on the cardiac signal and generate the risk stratification indicator based on the BRS measurement and the NSVT measurement.

19. The IMD of claim 16, wherein the processor is configured to:
determine an R−R interval difference indicating a time between successive ventricular depolarizations based on the cardiac signal;
determine a blood pressure difference based on the blood pressure signal; and
obtain the BRS measurement based on the R−R interval difference and the blood pressure difference.

20. The IMD of claim 19, wherein the blood pressure difference is a right ventricular blood pressure difference.

21. The IMD of claim 12, further comprising a telemetry module, wherein the processor is configured to communicate the implantation indicator to an external device via the telemetry module.

22. A system comprising:
an implantable medical device (IMD) configured to obtain a baroreflex sensitivity (BRS) measurement for a patient; and
an external computing device that receives the BRS measurement from the IMD, generates a risk stratification indicator based on the BRS measurement, wherein the risk stratification indicator classifies the patient into one of a plurality of cardiac arrhythmia or cardiac mortality risk categories.

23. The system of claim 22, wherein the external computing device generates an implantation indicator based on the risk stratification indicator that indicates the patient is a candidate for an implantable therapy device.

24. The system of claim 23, wherein the external computing device generates based on the risk stratification indicator an implantation indicator that indicates the patient is a candidate for an implantable cardioverter-defibrillator.

25. The system of claim 22, wherein the risk stratification indicator comprises an indicator of at least one of low risk, medium risk, and high risk of cardiac arrhythmia or cardiac mortality.

26. The system of claim 22, wherein the IMD comprises one or more electrode, a pressure sensor, and a processor, wherein the processor senses a cardiac signal via the one or more electrode and a blood pressure signal via the pressure sensor, and wherein the processor determines the BRS measurement from the cardiac signal and the blood pressure signal.

27. The system of claim 26, wherein the processor is further configured to determine a heart rate variability (HRV) measurement based on the cardiac signal and transmit the HRV measurement to the external computing device via the telemetry module, and wherein the external computing device is configured to generate the risk stratification indicator based on the BRS measurement and the HRV measurement.

28. The system of claim 26, wherein the processor is further configured to determine a non-sustained ventricular tachycardia (NSVT) measurement based on the cardiac signal and transmit the NSVT measurement to the computing device via the telemetry module, and wherein the external computing device is configured to generate the risk stratification indicator based on the BRS measurement and the NSVT measurement.

29. The system of claim 26, wherein the processor is configured to:
determine an R−R interval difference indicating a time between successive ventricular depolarizations based on the cardiac signal;
determine a blood pressure difference based on the blood pressure signal; and
determine the BRS measurement based on the R−R interval difference and the blood pressure difference.

30. The system of claim 29, wherein the blood pressure difference is a right ventricular blood pressure difference.

31. The system of claim 22, wherein the IMD further comprises a telemetry module, and wherein the processor transmits the BRS measurement to the external computing device via the telemetry module.

32. A computer readable medium comprising instructions that cause a programmable processor to:
receive a cardiac signal of a patient and a blood pressure signal of the patient via a measurement unit of an implantable medical device (IMD);
determine a baroreflex sensitivity (BRS) measurement based on the cardiac signal and the blood pressure signal; and
generate a risk stratification indicator based on the BRS measurement, wherein the risk stratification indicator classifies the patient into one of a plurality of cardiac arrhythmia or cardiac mortality risk categories.

33. The computer readable medium of claim 32, wherein the instructions cause the processor to generate based on the risk stratification indicator an implantation indicator that indicates the patient is a candidate for an implantable therapy device.

34. The computer readable medium of claim 33, wherein the implantation indicator indicates the patient is a candidate for an implantable cardioverter-defibrillator.

35. The computer readable medium of claim 32, wherein the instructions that cause the programmable processor to obtain the BRS measurement comprise instructions that cause the programmable processor to:
determine an R−R interval difference indicating a time between successive ventricular depolarizations based on the cardiac signal;
determine a blood pressure difference based on the blood pressure signal; and
determine the BRS measurement based on the R−R interval difference and the blood pressure difference.

36. The computer-readable medium of claim 35, wherein the blood pressure difference is a right ventricular blood pressure difference.

37. An implantable medical device (IMD) comprising:
means for obtaining a baroreflex sensitivity (BRS) measurement for a patient; and
means for generating a risk stratification indicator based on the BRS measurement, wherein the risk stratification indicator classifies the patient into one of a plurality of cardiac arrhythmia or cardiac mortality risk categories.

38. The IMD of claim 37, further comprising means for generating based on the risk stratification indicator an implantation indicator that indicates the patient is a candidate for an implantable therapy device.

39. The IMD of claim 37, further comprising means for detecting a cardiac signal and means for detecting a blood pressure signal, and wherein the means for obtaining the BRS measurement determines the BRS measurement based on the cardiac signal and the blood pressure signal.

40. The system of claim 39, wherein the blood pressure signal is a right ventricular blood pressure signal.
